(19) United States
(12) Patent Application Publication
(10) Pub. No.: US 2008/0220446 A1
(43) Pub. Date: Sep. 11, 2008

(54) Method for Detecting and/or Removing Protein and/or Peptide Comprising a Cross-Beta Structure From an Aqueous Solution Comprising a Protein

(76) Inventors: Martijn Frans Ben Gerard Gebbink, Eemnes (NL); Barend Bouma, Houten (NL)

Correspondence Address: TRASK BRITT, P.O. BOX 2550, SALT LAKE CITY, UT, 84110, US

(21) Appl. No.: 11/995,481

(22) PCT Filed: Jul. 13, 2006
(86) PCT No.: PCT/NL06/00363

§ 371 Date: Mar. 25, 2008

(30) Foreign Application Priority Data
Jul. 13, 2005 (US) 11181012

(51) Int. Cl. G01N 33/53; G01N 33/536; C07K 1/00; C07K 14/00

(52) U.S. Cl. 435/7.2; 436/536; 435/7.8; 530/412; 530/350

(57) ABSTRACT

The invention relates to the field of aqueous solutions comprising a protein. More specifically, the invention relates to the detection and/or removal of conformationally altered proteins and/or peptides comprising a cross-β structure from an aqueous solution comprising a protein. The invention provides methods for detecting and/or removing proteins and/or peptides comprising a cross-β structure from an aqueous solution comprising a protein, said method comprising contacting said aqueous solution comprising a protein with at least one cross-β structure-binding compound resulting in a bound protein or peptide with cross-β structure. The invention further provides a aqueous solution comprising a protein obtainable by a method of the invention, and a kit for carrying out the methods of the invention.

The invention relates to the field of aqueous solutions comprising a protein. More specifically, the invention relates to the detection and/or removal of conformationally altered proteins and/or peptides comprising a cross-β structure from an aqueous solution comprising a protein.

A protein or peptide is generally exposed to environmental influences which alter the original conformation and are therefore detrimental to said protein or peptide. Such environmental influences for example comprise temperature, light, pressure, humidity, enzymatic and microbial processes, pH and osmolarity of the solution, etc.

The present invention discloses that partially unfolded and/or misfolded proteins or peptides that are for example proteolysed, denatured, partially unfolded, glycated, oxidized, acetylated, multimerized or otherwise structurally altered, adopt a cross-β structure conformation. Furthermore, the present invention discloses that unwanted side effects and decreased specific activity are caused by proteins adopting a cross-β structure conformation. The presence of a cross-β structure is therefore indicative for degraded and/or denatured and/or multimerized protein, peptide and/or polypeptide.

The terms unfolding, refolding and misfolding relate to the three-dimensional structure of a protein or peptide. Unfolding means that the protein or peptide loses the three-dimensional structure and takes a linear arrangement. The term refolding relates to the coiling back into the original three-dimensional structure. By refolding, a protein can regain its native configuration, or an incorrect refolding can occur. Said incorrect refolding is also called misfolding. During unfolding and refolding, the formation of cross-β structures can occur.

The present invention discloses methods and means for detecting said cross-β structures in proteins, peptides and/or polypeptides, preferably in an aqueous solution. The present invention also discloses methods for removing said cross-β structures from proteins, and/or peptides, preferably in an aqueous solution. The methods of the invention are suitable for diminishing the unwanted side effects and the toxicity of said proteins, peptides and/or polypeptides, and for increasing the specific activity of said proteins, and/or peptides.

Generally, the specific activity of said protein, peptide and/or polypeptide in a solution is decreased after formation of cross-β structures. In addition, said cross-β structure in turn increases the formation of more cross-β structures in the protein and/or peptides, thereby increasing the degradation and/or denaturation and/or refolding and/or multimerization. Furthermore, the presence of a cross-β structure in a protein, peptide and/or polypeptide increases the risk of toxic and other unwanted side effects when said protein, peptide and/or polypeptide is administered to a subject, said subject being an animal or a human.

Many proteins, peptides or polypeptides used by man for different purposes are either derived from natural sources such as animals or plants, or they are synthesized or produced in vitro. Proteins, peptides or polypeptides are for example also used in the preparation of products like, food products, base products for the production of food, detergents, preferably detergents comprising enzymes, and/or cosmetic products. Said proteins, peptides or polypeptides are also used for diagnostic purposes like for example antisera and antigen preparations. In yet another embodiment, said proteins, peptides or polypeptides are used in analytical or biochemical chemistry, for example as commercially available biochemical base compounds such as proteins and enzymes, preferably in purified form.

Quality aspects are of great concern with any production and/or purification process comprising a protein, peptide or polypeptide. Protein stability during production, purification and storage is therefore important to manufacturers and to customers. Yet, in spite of these concerns, a number of accepted treatments of proteins may alter the conformation of said protein and therefore, induce cross-β structures in said protein.

For example, one generally accepted way of stabilizing a protein is by freezing said protein below zero degrees Celsius. Freezing and thawing may severely affect the conformation of proteins, peptides and/or polypeptides. Another accepted method of preservation is lyophilization. With this method, a protein is freeze-dried by evaporation of the aqueous solution below zero degrees Celsius. Many proteins and/or peptides are stored and sold in a dry form as lyophilized protein. Reconstitution of said lyophilized protein or peptide with a suitable aqueous solution is generally performed before the protein is used. Both the freezing and evaporation step, but also the reconstituting step, comprise risks for conformational changes of the protein and/or peptide and the formation of cross-β structures in said protein and/or peptide. It is for example known that lyophilized proteins comprise a higher β-sheet content than their solubilized counterparts, indicative for a refolding process due to the treatment.

A protein and/or peptide in this specification comprises any protein and/or peptide that is capable of forming a cross-β structure. Alteration of the protein and/or peptide comprises for example denaturation, proteolysis, acetylation, glycation, oxidation or unfolding of proteins¹⁻⁴.

An increasing body of evidence shows that the partial or complete unfolding of initially properly folded native proteins leads to the formation of toxic structures in said proteins¹⁻⁴. The invention further discloses that said partial unfolding results in the formation of cross-β structures in a protein and/or peptide.

A cross-β structure is defined as a part of a protein or peptide, or a part of an assembly of peptides and/or proteins, which comprises an ordered group of β-strands, typically a group of β-strands arranged in a β-sheet, in particular a group of stacked β-sheets, also referred to as “amyloid”. A typical form of stacked β-sheets is in a fibril-like structure in which the β-sheets may be stacked in either the direction of the axis of the fibril or perpendicular to the direction of the axis of the fibril. Of course the term peptide is intended to include oligopeptides as well as polypeptides, and the term protein includes proteins with and without post-translational modifications, such as glycosylation and glycation. It also includes lipoproteins and complexes comprising proteins, such as protein-nucleic acid complexes (RNA and/or DNA), membrane-protein complexes, etc.

Different fluorescent light emission profiles of amyloid dyes, such as for example Congo red or Thioflavin T in staining various amyloid-like proteins indicate that different forms of cross-β structures occur. Said cross-β structures are for example found in glycated proteins and in fibrils⁵. Such fibrillar aggregates accumulate in various tissue types and are associated with a variety of degenerative diseases. The term “amyloid” is being used to describe fibrillar deposits (or plaques)⁶. In literature, an amyloid fibril is preferably defined as an aggregate that is stained by Congo red and/or Thioflavin T, that appears as fibrils under an electron microscope, and that contains an increased amount of β-sheet secondary structure⁶. Additionally, the presence of β-sheet rich structures can be defined with X-ray fibre diffraction techniques and/or Fourier transform infrared spectroscopy. A common denominator of amyloid-like structures is the presence of the cross-β structure structural element. Peptides or proteins with amyloid-like structures are cytotoxic to cells⁷⁻¹¹.

Diseases characterized by amyloid are referred to as conformational diseases or amyloidoses and include for example Alzheimer's disease (AD), light-chain amyloidosis, type II diabetes and spongiform encephalopathies like for example Bovine Spongiform Encephalopathy (BSE) and Creutzfeldt-Jakob's disease.

In addition, deleterious effects of aggregated proteins are not solely mediated by said amyloid fibrillar depositions of proteins, but also by soluble oligomers of aggregates with amyloid-like properties and by diffuse amorphous aggregates^{7,129,13,14}. The recent finding that toxicity is an inherent property of misfolded proteins implies a common mechanism for said conformational diseases^{5,7,10}.

The compounds listed in Table 1 and the proteins listed in Table 2 all bind to polypeptides with cross-β structure. In literature, this fold has been designated as protein aggregates, amorphous aggregates, amorphous deposit, tangles, (senile) plaques, amyloid, amyloid-like protein, amyloid oligomers, amyloidogenic deposits, cross-β structure, β-pleated sheet, cross-β spine, denatured protein, cross-β sheet, β-structure rich aggregates, infective aggregating form of a protein, unfolded protein, amyloid-like fold/conformation and perhaps alternatively. The common theme amongst all polypeptides that are ligands for one or more of the compounds listed in Table 1 and 2, is the presence of a cross-β structure.

The compounds listed in Table 1 and 2 are considered to be only an example of the compounds known to day to bind to proteins or peptides with cross-β structures. The lists are thus non-limiting. More compounds are known today that bind to amyloid-like protein conformation and are thus functional equivalents of the compounds in Table 1, 2, or 3. For example, in patent AU2003214375 it is described that aggregates of prion protein, amyloid, and tau bind selectively to polyionic binding agents such as dextran sulphate or pentosan(anionic), or to polyamine compounds such as poly(Diallyldimethylammonium Chloride) (cationic). Compounds with specificity for proteins and peptides with cross-β structure listed in this patent and elsewhere are equally suitable for methods and devices disclosed in this patent application. Moreover, also any compound or protein related to the ones listed in Table 1 and 2 are covered by the claims. For example, point mutants, fragments, recombinantly produced combinations of cross-β structure binding domains and deletion- and insertion mutants are part of the set of compounds as long as they are capable of binding to protein with a cross-β structure (i.e. as long as they are functional equivalents) In addition, any small molecule or protein that exhibits affinity for the cross-β structure can be used in any one of the methods and applications disclosed here.

The compounds listed in Table 3 are also considered to be part of the ‘Cross-β structure pathway’, and this consideration is based on literature data that indicates interactions of the listed molecules with compounds that likely comprise a cross-β structure but that have not been disclosed as such.

Generally, for the production of a protein and/or peptide, said protein and/or peptide is subjected to a number of processes like for example a synthesis process or an isolation process. Peptide synthesis processes are generally performed in a plant cell, a yeast cell or a bacteria, or a cell of an animal. A protein and/or peptide manufacturing process also comprises coupling of chemical molecules to a peptide or protein. Further, said protein and/or peptide is subjected to an isolation procedure or a purification procedure, and/or a concentrating process, like for example the isolation of recombinant protein from a bacterial production cell, or purification by a physical, or a chemical, or an immunological isolation method, and/or a formulation and/or a storage process, including for example a lyophilization process and/or the addition of a suitable stabilizer, a diluent and/or an adjuvant.

Any one of these processes affects the folding of a protein and/or peptide. Quality control in a manufacturing process preferably aims at identifying and/or minimizing the deleterious effects of each process step for a protein and/or peptide, thereby increasing the activity of the composition in the final product or composition and/or decreasing the undesired side effects of the composition.

Alteration of a protein and/or peptide is generally detected by two methods. The first method comprises measuring the amount of a specific binding site of protein and/or peptide. The second method comprisesmeasuring an increase in size or multimerization state of said protein and/or peptide.

As to the first of said methods, a partially unfolded or misfolded protein can still expose a specific binding site. Therefore, testing the quality of a protein and/or peptide by only testing for a specific binding site is not always a reliable method, because the partial unfolding or degradation of said protein and/or peptide is not detected.

The second of said methods, the size-related detection method is based on the concept that denaturation leads to aggregation of proteins, thereby increasing the size of the protein and/or peptide. One of several methods for detecting an increase in size of proteins is called size exclusion chromatography. Nowadays, size exclusion chromatography is widespread used as a method to analyse the contents of a protein composition. This technique is generally accepted for the testing of protein composition. (http://etd.utmem.edu/WORLD ACCESS/ymi/reviewofanalyticmethod.htm).

Because said detection method only detects the size of a protein and/or peptide, it cannot detect misfolded proteins or proteins with increased content of cross-β structures that have not aggregated or increased in size. Therefore, both methods have disadvantages and quality control based on both the above-described methods, does not prevent undesired side effects caused by conformational changes such as for example cross-β structures formed upon denaturation, proteolysis, chemical modification, or unfolding of proteins, in the absence of increased molecular size. Moreover, nowadays guidelines that determine the acceptable amounts of aggregates of protein and/or peptide in solutions are based on technical limitations of the available purification methods, rather than on knowledge about expected undesired side effects of the aggregated proteins. Therefore an improved quality control method is needed by scientists involved in development of protein and/or peptide production and formulations and for manufacturers of compositions comprising protein and/or peptide.

The present invention provides such an improved method to detect the presence of cross-β structure in a protein and/or peptide in an aqueous solution. The invention provides also methods for the removal of proteins or peptides comprising a cross-β structure conformation, thereby reducing the unwanted side effects and toxicity and increasing the specific activity per gram protein of said compositions. Therefore, the methods of the invention provide a person skilled in the art with a method of monitoring and optimising the production methods and storing conditions of a protein and/or peptide in an aqueous solution.

In one embodiment, the present invention discloses a method for detecting a protein and/or peptide comprising a cross-β structure in an aqueous solution comprising a protein and/or peptide, said method comprising, contacting said aqueous solution comprising a protein with at least one cross-β structure-binding compound resulting in a bound protein and/or peptide comprising a cross-β structure and, detecting whether bound protein and/or peptide comprising cross-β structures are present in said aqueous solution. Binding of one or more of the cross-β structure-binding compounds to a cross-β structure is detected by means of a visualization reaction as for example by fluorescent staining or an enzymatic or calorimetric detection, or by any other visualization system available to a skilled person. The specification provides a number of methods for detecting the bound protein and/or peptide comprising cross-β structures and also methods for determining the amount of bound protein and/or peptide comprising cross-β structures.

The invention discloses that various molecules or compounds, as described in Table 1, 2 and/or 3 of the application, alone or in combination with each other or other binding compounds, are capable of binding to a protein with a cross-β structure or a part of a protein and/or peptide essentially only comprising a cross-β structure. The term cross-β structure and cross-β structure conformation both refer to a three dimensional structure in a protein characterized by the presence of stacked or layered cross-β sheets; the terms are used interchangeably herein.

Therefore, the specification discloses a number of cross-β structure-binding compounds, with which the methods of the invention can be performed. Therefore, in another embodiment, the invention provides a method according to the invention, wherein said cross-β structure-binding compound is a compound according to table 1, or table 2, or table 3 or a functional equivalent of any of said compounds. A functional equivalent of a cross-β structure-binding compound is a compound which is capable of binding to a cross-β structure.

In Table 1, 2 and/or 3, various different compounds are described that bind to compounds with a cross-β structure. For example, Table 1 comprises among other, dyes like Thioflavin T, Thioflavin S, and Congo Red, that are used for staining amyloid molecules in histological sections or in solution. Table 2 comprises bioactive compounds binding to compounds comprising cross-β structures such as tissue-type plasminogen activator (tPA), factor XII, fibronectin, and others.

In Table 3, proteins are disclosed that are involved in the cross-β structure pathway, like for example, antibodies, heat shock proteins and receptors.

The invention also provides a protein specific way of detecting and removing protein and/or peptide comprising cross-β structures, by combining the protein specific binding of an antibody or a functional part thereof (i.e. a part that binds specifically to a protein), with a cross-β structure binding compound. Therefore, the invention also provides molecular recognition units binding to compounds with cross-β structures, or single chains of antibodies. The invention further provides bi-specific recombinant binding molecules for example comprising the binding portion of tPA and an antibody, or the binding portion of a bioactive compound binding to proteins with cross-β structures with the binding part of an antibody.

Because of the unwanted side effects, the decrease in specific activity of a protein, and the toxicity for cells and organisms, it is preferred to know whether a protein and/or peptide comprises cross-β structures. It is disclosed in the specification how to detect a protein and/or peptide comprising a cross-β structure in an aqueous solution. Said aqueous solution comprises a protein, a detergent enzyme, a food and/or a food supplement, a commercially available protein, blood and/or blood products, a cosmetic product, and/or a cell. Said protein is for example a product comprising an enzyme for baking bread or brewing beer, or stabilizing food products. Said solution also comprises for example enzymes used for the production of base products. Said solution also comprises for example milk and milk products and pastes used in food production, for example meat paste, or specific protein compositions such as for example lubricants. Said solution also comprises for example tissue culture fluid, for example from recombinant production systems with prokaryotic or eukaryotic cells or from cell-free production system for recombinant production of proteins or peptides.

A person skilled in the art can now use the methods of the invention or modifications thereof to detect or deplete or detect and deplete proteins and/or peptides comprising cross-β structure from any aqueous solutions comprising a protein and/or peptide. For example, any of the compounds listed in Table 1, 2 or 3 can be used to detect cross-β structure in for example aqueous solutions that are intended for use in laboratory, for example for tissue culture, biochemistry, crystallization and so on. For example the function of a protein can be studied before and after detection and depletion of proteins or peptides with cross-β structure. Furthermore, the present invention discloses methods to induce cross-β structure in a known protein, then select suited cross-β structure-binding compounds to said altered protein comprising a cross-β structure, and then use said binding compounds for purifying the protein product of a synthesizing and/or purification method.

Therefore, the present invention in another embodiment discloses a method for controlling a manufacturing process, and/or storage process of an aqueous solution comprising a protein, said method comprising, contacting said aqueous solution with at least one cross-β structure-binding compound resulting in a bound cross-β structure and, detecting whether bound cross-β structures are present in said aqueous solution at various stages of said manufacturing and/or storage process.

After detection of cross-β structures in a solution by a cross-β structure-binding compound, the same reaction, or optionally another reaction with a cross-β structure-binding compound is suitable for removing the cross-β structures from the solution. The bound proteins and/or peptides comprising a cross-β structure are removed by binding the cross-β structure-binding compounds to at least one other binding molecule that is bound to a solid phase, or to a third binding compound. Therefore, the invention in another embodiment discloses a method for removing a cross-β structure from an aqueous solution comprising a protein, said method comprising, contacting said aqueous solution with at least one cross-β structure-binding compound resulting in bound proteins and/or peptides comprising a cross-β structure and, allowing binding of said proteins and/or peptides comprising a cross-β structure to said cross-β structure-binding compound and, separating said bound proteins and/or peptides comprising a cross-β structure from said aqueous solution.

It is disclosed herein that the compounds of Table 1, 2 and/or 3 of the application are suitable cross-β structure-binding compounds. Therefore, the present invention discloses a method according to the invention, wherein said cross-β structure-binding compound is a compound according to table 1, or table 2, or table 3 or a functional equivalent of any of said compounds.

For efficient removal of bound proteins and/or peptides comprising a cross-β structure, a cross-β structure-binding compound is attached to another binding compound or to a solid phase by chemical or physical methods.

As a solid phase, many materials are suitable for binding a cross-β structure-binding compound, such as for example, glass, silica, polystyrene, polyethylene, polypropylene, nylon, vinyl, agarose/Sepharose beads, beads containing iron or other metals and so on. In one embodiment of the invention, said solid phase has the physical form of beads. In another embodiment said solid phase has the shape of a tube or a plate or a well in, for instance an ELISA plate, or a dipstick. Numerous binding techniques are available for coupling the cross-β structure-binding compounds to said solid phase, like for example, CyanogenBromide (CNBr), NHS, Aldehyde, epoxy, Azlactone, biotin/Streptavidin, antigen-antibody, and many others. The amount of bound protein and/or peptide comprising cross-β structures is measured for example by staining said cross-β structures and is a measure for the quality of the proteins and/or peptides in said solution.

It generally depends on the attachment method that is selected how and when the cross-β structure-binding compound is attached to another molecule or compound. For example, a preferred binding of said compound of Table 1 to another compound occurs before binding a protein and/or peptide comprising a cross-β structure, or more preferred during the process of said binding of a cross-β structure, or most preferred after binding of a protein and/or peptide comprising a cross-β structure. Therefore, the present invention discloses a method according to the invention, wherein said cross-β structure-binding compound is bound to a second compound before, during or after the binding of said cross-β structure-binding compound to a protein and/or peptide comprising a cross-β structure.

As described above, it depends on the attachment method and on the type of solid phase how and when the cross-β structure-binding compound and/or its second binding compound is attached to a solid phase. In one embodiment, the compound of Table 1, 2 or 3 is attached to a solid phase, and in another embodiment of the invention, said compound of Table 1, 2 or 3 or an equivalent thereof is first attached to a second binding compound, which in its turn is attached to a solid phase. Therefore, the present invention discloses a method according to the invention, wherein said second compound is bound to a solid face. For example said second compound comprises an antibody directed against part of a compound of Table 1, 2, or 3, or comprises a (chemical) linker that is capable of binding a compound of Table 1, 2, or 3. Although in many cases it will be enough to contact a protein and/or peptide comprising a cross-β structure with a cross-β structure-binding compound, or said complex with a second binding compound, it of course also within the scope of the present invention that the second binding compound is also capable of binding to a third binding compound or even to a fourth or fifth and so on. Therefore, the present invention in another embodiment discloses a method of the invention, wherein said cross-β structure binding compound, bound to a second compound is further bound to a third or fourth or further binding compound before, during or after the binding of said cross-β structure binding compound to a protein and/or peptide comprising a cross-β structure. In a preferred embodiment a second, third, or fourth, or further binding compound. is bound to a solid phase. Therefore, the present invention also discloses a method, wherein said third or fourth compound is bound to a solid phase. In another embodiment of the invention, said continued binding of more binding molecules induces the formation of aggregates, for example by agglutination, that do not need a further solid phase to be separated from the aqueous solution.

The methods of the invention are useful for controlling the different stages of a manufacturing process of a protein and/or peptide. In general, the specification of a process for manufacturing a composition comprising a protein and/or peptide is described in a handbook according to good manufacturing practice (GMP) and good laboratory practice (GLP). GLP and GMP quality control is a valuable tool for manufacturers of protein compositions and for manufacturers of constituents comprising a protein and/or peptide and it helps and enables them to produce products of a steady quality and to increase the quality by monitoring the manufacturing and storage process. The present invention discloses methods that help manufacturers to detect compounds with cross-β structures in the product. A qualitative difference is thus made between products with cross-β structures or products without cross-β structures, or with low levels of cross-β structure. By monitoring the processes with methods of the invention, manufacturers are capable of omitting processes or chemicals or physical conditions or circumstances or treatments that induce the formation of cross-β structures, and it enables them to select processes or chemicals or circumstances that do not induce cross-β structures and/or raise the level of cross-β structures in a solution comprising a protein.

The present invention also discloses a method for decreasing and/or preventing undesired side effects of an aqueous solution comprising a protein and/or increasing the specific activity per gram protein of an aqueous solution, said method comprising detecting and removing any unfolded protein or peptide and/or aggregated protein or peptide and/or multimerized protein or peptide comprising a cross-β structure from said aqueous solution according to any method of the invention.

In one preferred embodiment, the present invention discloses a method for detecting and/or measuring a cross-β structure-inducing ability of a solid surface, by contacting said surface with a protein and detecting denatured protein by subsequently contacting said surface with a cross-β structure-binding compound. With said method of the invention, a person skilled in the art is capable of selecting materials for a container for storing protein. The same procedure is suitable for selecting a reaction vessel, a production vessel, a storage vessel and/or a tube connecting said vessels. The above-described method is also suitable for detecting and/or measuring a cross-β structure-inducing ability of a molecule, for example of a salt, or a dye, or an enzyme, or a chemical compound such as for example alcohol or formaldehyde or glucose. Therefore, the present invention discloses in another embodiment a method for detecting and/or measuring a cross-β structure-inducing ability of a substance, by contacting said substance with a protein and detecting denatured protein by subsequently contacting said molecule and/or said protein with a cross-β structure binding compound. Substances that have the ability to induce a cross-β structure are then removed or avoided in the production, purification and storage of a protein. Therefore, the present invention enables a person skilled in the art to avoid the use of substances as a part of the aqueous solution or as a part of a wall of a container for production, purification, or storage of said protein and/or peptide. In another embodiment, the invention teaches the person skilled in the art to avoid substances inducing cross-β structure in the preparation of a solution comprising a protein and/or peptide. Therefore, the present invention provides a method for selecting substances for production and/or dilution, and/or preservation of a composition comprising a protein and/or peptide.

In yet another embodiment, the present invention discloses a method for detecting and/or measuring a cross-β structure-inducing ability of a physical condition such as for example, pH, pressure, temperature, salt concentration and/or protein concentration. A recombinant protein and/or peptide is subjected to various physical conditions and the increase or induction of the amount of cross-β structures is measured by contacting said protein and/or peptide with a cross-β structure-binding compound according to a method of the invention. Binding of a protein and/or peptide comprising a cross-β structure with a cross-β structure-binding compound is detected using the methods of the invention. The above-described method is a valuable tool for detecting cross-β structure-inducing circumstances during production, purification, and storage. Therefore, the present invention discloses a process to improve production, purification and storage of product comprising a protein and/or peptide.

Because the present invention discloses how to detect cross-β structures in an aqueous solution comprising a protein, a skilled person is able to select conditions that prevent or decrease the induction of cross-β structures during the synthesis or production or purification of a protein and/or peptide.

A protein and/or peptide, which is produced, processed or purified according to any one of the methods of the present invention, comprises less compounds with cross-β structures, and is therefore less toxic, thrombogenic, immunogenic, inflammatory or harmful for a mammal including a human after administration of said protein and/or peptide. Furthermore, because of the decreased presence of protein and/or peptide comprising cross-β structures, the purity and the specific activity of a protein is preferably higher per gram protein present in said protein, and therefore, less protein is needed to achieve the same pharmacological effect. A protein and/or peptide that is purified by any of the methods of the invention is therefore of higher quality, and exerts less side effects than a protein and/or peptide that is not purified. The difference between a protein and/or peptide according to the invention and a another protein and/or peptide is in the lower amount of protein and/or peptide comprising cross-β structures that is detectable in the protein and/or peptide according to the invention.

Therefore, the present invention in another embodiment provides an aqueous solution comprising a protein and/or peptide, obtainable by a method according to the invention.

In another embodiment, the specification provides a kit of parts, comprising for example one or more cross-β structure binding compounds as depicted in Table 1, or 2, or possibly 3, and optionally one or more compounds binding said cross-β structure binding compound, and a means for detecting bound protein and/or peptide comprising a cross-β structure as described elsewhere in this specification, thereby making the kit suitable for carrying out a method according to the invention such as for example detecting protein and/or peptide comprising a cross-β structures, and or removing protein and/or peptide comprising a cross-β structures from a protein solution. Therefore, the present invention provides a kit for carrying out a method according to the invention, comprising all necessary means for binding a protein or peptide comprising a cross-β structure to a cross-β structure-binding compound, and/or removing a protein or peptide comprising a cross-β structure from an aqueous solution comprising a protein and/or peptide.

The presence of bound proteins or peptides with cross-β structures is in another embodiment detected by an enzymatic assay. As an example of an enzymatic assay the specification provides tPA+plasminogen+plasmin substrate S-2251 (Chromogenix Spa, Milan, Italy) in a suitable buffer. Preferably the buffer is HBS (10 mM HEPES, 4 mM KCl, 137 mM NaCl, pH 7.3). Standard curve is made with a control with a cross-β structure. Titration curves are made with a sample before and after a treatment/exposure to a putatively denaturing condition. Alternatively the detection of bound cross-β structures is achieved by a test wherein factor XII with activated factor XII substrate S-2222 or S-2302 is present in a suitable buffer. Preferably, the buffer is 50 mM, 1 mM EDTA, 0.001% v/v Triton-X100. Standard curves are made with known cross-β structure rich activators of factor XII; preferably dextran sulphate 500,000 k (DXS500k) with a protein; preferably the protein is endostatin or albumin; preferably with glycated haemoglobin, Aβ, amyloid fibrin peptide NH₂-148KRLEVDIDIGIRS160-COOH with K157G mutation. In yet another embodiment, the presence of bound cross-β structures is detected by a test comprising factor XII with prekallikrein and high molecular weight kininogen and either substrate Chromozym-PK for kallikrein or a substrate for activated factor XII in a suitable buffer; preferably HBS. Standard curves are made with known cross-β structure rich activators of factor XII; preferably DXS500k or kaolin with a protein; preferably the protein is endostatin or albumin; preferably with glycated haemoglobin, Aβ, amyloid fibrin peptide NH₂-148KRLEVDIDIGIRS160-COOH with K157G mutation.

The specification provides in one embodiment of a kit for example a filter-like element, said element capable of binding protein and/or peptide comprising cross-β structures or binding cross-β structure binding compounds. Said filter is used to pass a solution comprising a protein and/or peptide through it. In another embodiment, said filter is used in the production or packaging of a protein and/or peptide. In another embodiment, the kit of the specification provides an ELISA plate, or a dipstick for detecting protein and/or peptide comprising cross-β structures in a composition comprising a protein and/or peptide or a filtration device for removing protein and/or peptide comprising cross-β structures from a solution comprising a protein, peptide, or polypeptide.

After removal of the protein and/or peptide comprising cross-β structures from a composition comprising a protein and/or peptide, the resulting composition is tested again to control whether the amount of protein and/or peptide comprising cross-β structures in said composition has actually decreased.

The cross-β structure-binding compounds of the invention are also suitable for detecting a cell with cross-β structures on the surface. A cell with cross-β structures on the surface is for example a bacterial cell, or a yeast cell or a eukaryotic cell. In biotechnological protein production systems, use is made of bacterial cells or yeast cells or eukaryotic cells to produce protein. Selection of those cell types that have less cross-β structures on the surface than other cells is advantageous for a production system, because induction of cross-β structures in the produced protein is less. Therefore, in another embodiment, the present invention also provides a method for detecting a cell comprising a cross-β structure on its surface in a collection of cells, said method comprising contacting said cell with a cross-β structure-binding molecule, and measuring binding of said molecule to said cell.

In a preferred embodiment, a collection of cells is made better suited for production of protein by removing cells with cross-β structures on the surface. Said removing is achieved using the cross-β structure-binding compounds of the invention. Therefore, the present invention in another embodiment discloses a method for removing a cell comprising a cross-β structure on its surface from a collection of cells, said method comprising contacting said cell with a cross-β structure-binding molecule, and binding said molecule to a solid surface.

Examples of useful applications of a method according to the invention are provided above and even more examples are provided below. In general it can be said that if one wants to study or obtain a protein with a particular property, it is important to check each and every treatment on their cross-β structure inducing capabilities on said protein. If for example a protein is used in the food industry or as a biochemical compound in research (for example biomedical research, or in diagnostics it is important to check the production, purification and storage conditions. If one wants to study the activity of a protein (for example an enzyme) it is important to study all the conditions to which such a protein is subjected.

Other, non-limiting, applications of a method according to the invention are
testing of conditions for producing, purifying and storing proteins used for growing crystals for protein crystallography purposes; some of the presently used conditions result in the formation of cross-β structure in a protein and hence hamper the growth of high-quality crystals of said protein; conditions (to be) used in crystallography are now tested for their cross-β structure inducing capability and a selection is made for conditions that do not or only slightly induce the formation of cross-β structure in a protein;
testing of chemical/biochemical/biophysical conditions used in protein purifications; independent of the source of protein (naturally expressed or recombinantly expressed) proteins are typically subjected to one or multiple purification steps to obtain high grade preparations comprising a protein and/or peptide. All treatments performed with a protein or peptide in such purifications, such as buffer composition, temperature, column material, dialysis membranes, membranes used for concentration, is checked with a method according to the invention and conditions are selected that do not or only slightly induce cross-β structure formation in the to be purified protein;
testing of conditions and/or solutions for protein refolding from an aggregated state to a native fold; independent of the source of the protein with non-native fold (naturally expressed or recombinantly expressed; for example Escherichia coli inclusion bodies), proteins are typically subjected to exposure to one or more solutions that putatively aid the folding from a non-native fold to a native fold. The solutions are now checked with a method according to the invention for their propensity to induce the cross-β structure in proteins by testing the content of cross-β structure in the proteins after the exposure to the solutions. Solutions can now be selected that do not result in cross-β structure and thus may aid the adoption of a native fold.
selection and development of cell culture solutions or laboratory liquid equipment comprising proteins or peptides in general.

It is revealed in the specification that several physical/chemical conditions influence the fold of a protein. Exposure to CL or DXS500k, a freeze-thaw cycle, variations in protein purification protocol, heating, change in pH, the source of the protein and exposure to plastic all introduce a structural rearrangement in the protein accompanied by the formation of the amyloid-like cross-β structure fold. This new fold can be detected by, amongst others, tPA binding, tPA activation, factor XII binding and by conventional amyloid fluorescence assays.

In another embodiment, the present invention discloses a method according to the invention, wherein said cross-β structure binding compound comprises ellagic acid.

The invention is further explained in the examples, without being limited by them.

TABLE 1
cross-β structure binding compounds

| Congo red | Chrysamine G | Thioflavin T |
|---|---|---|
| 2-(4′-(methylamino)phenyl)-6- | Any other amyloid- | Glycosaminoglycans |
| methylbenzothiaziole | binding | |
| | dye/chemical | |
| Thioflavin S | Styryl dyes | BTA-1 |
| Poly(thiophene acetic acid) | conjugatede | Ellagic acid |
| | polyeclectrolyt | |
| | PTAA-Li | |

TABLE 2
Proteins that bind to and/or interact with misfolded proteins and/or with
proteins comprising cross-β structure

| | | |
|---|---|---|
| Tissue-type plasminogen | Finger domain(s) of tPA, factor | Apolipoprotein E |
| activator | XII, fibronectin, HGFA | |
| Factor XII | Plasmin(ogen) | Matrix metalloprotease-1 |
| Fibronectin | 75 kD-neurotrophin receptor | Matrix metalloprotease-2 |
| | (p75NTR) | |
| Hepatocyte growth factor | α2-macroglobulin | Matrix metalloprotease-3 |
| activator | | |
| Serum amyloid P component | High molecular weight kininogen | Monoclonal antibody |
| | | 2C11(F8A6)^{‡} |
| C1q | Cathepsin K | Monoclonal antibody 4A6(A7)^{‡} |
| CD36 | Matrix metalloprotease 9 | Monoclonal antibody 2E2(B3)^{‡} |
| Receptor for advanced glycation | Haem oxygenase-1 | Monoclonal antibody 7H1(C6)^{‡} |
| endproducts | | |
| Scavenger receptor-A | low-density lipoprotein receptor- | Monoclonal antibody 7H2(H2)^{‡} |
| | related protein (LRP, CD91) | |
| Scavenger receptor-B | DnaK | Monoclonal antibody 7H9(B9)^{‡} |
| ER chaperone Erp57 | GroEL | Monoclonal antibody 8F2(G7)^{‡} |
| Calreticulin | VEGF165 | Monoclonal antibody 4F4^{‡} |
| Monoclonal conformational | Monoclonal conformational | Amyloid oligomer specific |
| antibody WO1 (ref. (O'Nuallain | antibody WO2 (ref. (O'Nuallain | antibody (ref. (Kayed et al., |
| and Wetzel, 2002)) | and Wetzel, 2002)) | 2003)) |
| formyl peptide receptor-like 1 | α(6)β(1)-integrin | CD47 |
| Rabbit anti-albumin-AGE | CD40 | apo A-I belonging to small high- |
| antibody, Aβ-purified^{a)} | | density lipoproteins |
| apoJ/clusterin | 10 times molar excess PPACK, 10 mM | CD40-ligand |
| | εACA, (100 pM-500 nM) | |
| | tPA²⁾ | |
| macrophage scavenger receptor | broad spectrum (human) | BiP/grp78 |
| CD163 | immunoglobulin G (IgG) | |
| | antibodies (IgIV, IVIg) | |
| Erdj3 | haptoglobin | |

^{‡}Monoclonal antibodies developed in collaboration with the ABC-Hybridoma Facility, Utrecht University, Utrecht, The Netherlands.
^{a)}Antigen albumin-AGE and ligand Aβ were send in to Davids Biotechnologie (Regensburg, Germany); a rabbit was immunized with albumin-AGE, antibodies against a structural epitope were affinity purified using a column with immobilized Aβ.
²⁾PPACK is Phe-Pro-Arg-chloromethylketone (SEQ-ID 8), εACA is ε-amino caproic acid, tPA is tissue-type plasminogen activator

TABLE 3
Proteins that interact with amyloid-like misfolded protein

| | | |
|---|---|---|
| Monoclonal antibody 4B5 | Heat shock protein 27 | Heat shock protein 40 |
| Monoclonal antibody 3H7^{‡} | Nod2 (=CARD15) | Heat shock protein 70 |
| FEEL-1 | Pentraxin-3 | HDT1 |
| LOX-1 | Serum amyloid A proteins | GroES |
| MD2 | Stabilin-1 | Heat shock protein 90 |
| FEEL-2 | Stabilin-2 | CD36 and LIMPII analogous-I |
| | | (CLA-1) |
| Low Density Lipoprotein | LPS binding protein | CD14 |
| C reactive protein | CD45 | Orosomucoid |
| Integrins | alpha-1 antitrypsin | apo A-IV-Transthyretin |
| | | complex |
| Albumin | Alpha-1 acid glycoprotein | β2-glycoprotein I |
| Lysozyme | Lactoferrin | Megalin |
| Tamm-Horsfall protein | Apolipoprotein E3 | Apolipoprotein E4 |
| Toll-like receptors | Complement receptor | CD11d/CD18 (subunit aD) |
| | CD11b/CD18 (Mac-1, CR3) | |
| CD11b2 | CD11a/CD18 (LFA-1, subunit aL) | CD11c/CD18 (CR4, subunit |
| | | aX) |
| Von Willebrand factor | Myosin | Agrin |
| Perlecan | Chaperone60 | b2 integrin subunit |
| proteins that act in the | proteins that act in the | Macrophage receptor with |
| unfolded protein response | endoplasmic reticulum stress | collagenous structure |
| (UPR) pathway of the | response (ESR) pathway of | (MARCO) |
| endoplasmic reticulum (ER) | prokaryotic and eukaryotic cells | |
| of prokaryotic and eukaryotic | | |
| cells | | |
| 20S | chaperone 16 family members | HSC73 |
| HSC70 | translocation channel protein | 26S proteasome |
| | Sec61p | |
| 19S cap of the proteasome | UDP-glucose:glycoprotein | carboxy-terminus of |
| (PA700) | glucosyl transferase (UGGT) | chaperone70-interacting |
| | | protein (CHIP) |
| Pattern Recognition | Derlin-1 | Calnexin |
| Receptors | | |
| Bcl-2 asociated athanogene | GRP94 | Endoplasmic reticulum p72 |
| (Bag-1) | | |
| (broad spectrum) (human) | proteins that act in the | The (very) low density |
| immunoglobulin M (IgM) | endoplasmic reticulum associated | lipoprotein receptor family |
| antibodies | degradation system (ERAD) | |
| Fc receptor | | |

^{‡}Monoclonal antibodies developed in collaboration with the ABC-Hybridoma Facility, Utrecht University, Utrecht, The Netherlands.

### EXAMPLES

### Materials & Methods

Preparation of Cross-β Structure Rich Compounds

For preparation of advanced glycation end-product (AGE) modified bovine serum albumin, 100 mg ml⁻¹ of albumin was incubated with phosphate buffered saline pH 7.3 (PBS) containing 1 M of glucose-6-phosphate (g6p) and 0.05% m/v NaN₃, at 37° C. in the dark. Glycation was prolonged up to 23 weeks⁵. To prepare glycated haemoglobin (Hb-AGE), human haemoglobin (Hb, Sigma-Aldrich, H7379) at 5 mg ml⁻¹ was incubated for 32 weeks at 37° C. with PBS containing 1 M of g6p and 0.05% m/v of NaN₃. In control solutions, g6p was omitted. After incubations, solutions were extensively dialyzed against distilled H₂O and, subsequently, stored at 4° C. Protein concentrations were determined with advanced protein-assay reagent ADV01 (Cytoskeleton, Denver, Colo., USA). Glycation and formation of AGE was confirmed by measuring intrinsic fluorescent signals from AGE; excitation wavelength 380 nm, emission wavelength 435 nm. In addition, binding of AGE-specific antibodies was determined. Presence of cross-β structure or cross-β structure conformation in albumin-AGE was confirmed by enhancement of Congo red fluorescence, enhancement of Thioflavin T (ThT) fluorescence, the presence of β-sheet secondary structure, as observed with circular dichroism spectropolarimetry (CD) analyzes, and by X-ray fiber diffraction experiments⁵. Presence of cross-β structures in Hb-AGE was confirmed by tPA binding, CD analyses, transmission electron microscopy (TEM) imaging of fibrillar structures and by Congo red fluorescence measurements. Amyloid preparations of human γ-globulins were made as follows. Lyophilized γ-globulins (G4386, Sigma-Aldrich) were dissolved in a 1(:)1 volume ratio of 1,1,1,3,3,3-hexafluoro-2-propanol and trifluoroacetic acid and subsequently dried under an air stream. Dried γ-globulins were dissolved in H₂O to a final concentration of 1 mg ml⁻¹ and kept at room temperature for at least three days, or kept at 37° C. for three days and subsequently at −20° C. Aliquots were stored at −20° C. and analyzed for the presence of cross-β structures. Fluorescence of Congo red and ThT was assessed. In addition tPA binding was analyzed in an ELISA and tPA activating properties in a chromogenic plasminogen (Plg) activation assay. In addition, the macroscopic appearance of denatured γ-globulins was analyzed with TEM imaging.
Human amyloid-β (Ap) (1-40) Dutch type (DAEFRHDSGYEVHHQKLVFFAQDVGSNKGAIIGLMVGGVV,), was disaggregated in a 1:1 (v/v) mixture of 1,1,1,3,3,3-hexafluoro-2-isopropyl alcohol and trifluoroacetic acid, air-dried and dissolved in H₂O (10 mg ml⁻¹). After three days at 37° C., the peptide was kept at room temperature for two weeks, before storage at 4° C. Aβ solutions were tested for the presence of amyloid conformation by ThT or Congo red fluorescence and by TEM imaging. Negative control for cross-β structure detection assays was non-amyloid fragment FP10 of human fibrin α-chain(148-157) (KRLEVDIDIK)^{16,17}. FP10 was dissolved at a concentration of 1 mg ml⁻¹ in H₂O and stored at 4° C. This solution was used as a negative control for ThT fluorescence assays.

Cloning and Expression of Recombinant Fibronectin Type I Domains

F4-5 domains and the F domain of tPA with a carboxy-terminal His₆-tag were also expressed in Saccharomyces cerevisiae. The cDNA constructs were prepared following standard procedures¹⁸, by the Biotechnology Application Center (BAC-Vlaardingen/Naarden, The Netherlands). Domain boundaries of Fn F4-5 and tPA F were taken from the human Fn and human tPA entries in the Swiss-Prot database (P02751 for Fn, P00750 for tPA) and comprised amino-acids NH₂—I182-V276-COOH of Fn F4-5 and NH₂-G33-S85-COOH of tPA. Affinity purification of the expressed proteins was performed using His₆-tag-Ni²⁺ interaction and a desalting step. Constructs were stored at −20° C. in PBS pH 7.0. The molecular size of the constructs was checked on a Coomassie brilliant blue-stained SDS-PAGE gel.

Totally Chemical Synthesis of Fibronectin Type I Domains

Totally chemical synthesis of the F domains of hepatocyte growth factor activator (HGFA, SwissProt entry Q04756) and tPA (SwissProt entry P00750) was performed in the laboratory of Dr. T. M. Hackeng (Academic Hospital Maastricht, The Netherlands), according to standard procedures¹⁹. Both domains were synthesized as two separate peptides that were subsequently ligated using native chemical ligation. The tPA F domain was completed with a carboxy-terminal acetylated lysine residue or biotinylated lysine residue. The HGFA F domain was supplied with an acetylated lysine residue. Products were analyzed on a reversed phase HPLC column and with mass spectrometry.

Cloning, Expression and Purification of the Soluble Extracellular Domains of Receptor for Advanced Glycation Endproducts

The soluble extracellular part, of the receptor for AGE (sRAGE) was cloned, expressed and purified as follows (Q.-H. Zeng, Prof. P. Gros, Dept. of Crystal- & Structural Chemistry, Bijvoet Center for Biomolecular Research, Utrecht University, Utrecht, The Netherlands). Human cDNA of RAGE was purchased from RZPD (clone IRALp962E1737Q2, RZPD, Berlin, Germany). For PCRs, the gagatctGCTCAAAACATCACAGCCCGG forward primer was used comprising a BgIII site, and the gcggccgcCTCGCCTGGTTCGATGATGC reverse primer with a NotI site. The soluble extracellular part of RAGE comprises three domains spanning amino-acid residues 23-325. The PCR product was cloned into a pTT3 vector, containing an amino-terminal His-tag and a thrombin cleavage site. The sRAGE was expressed in 293E hamster embryonic kidney cells at the ABC-protein expression facility (Utrecht University, Utrecht, The Netherlands). Concentrated cell culture medium was applied to a Hi-trap Chelating HP Ni²⁺-NTA column (Amersham Biosciences Europe, Roosendaal, The Netherlands). The running buffer was 25 mM Tris-HCl, 500 mM NaCl, pH 8.0. The protein was eluted by using a step gradient of 0 to 500 mM imidazole. Purity of the His-sRAGE was depicted fiom Coomassie stained SDS-PAGE gels. After concentration, the buffer was exchanged to 20 mM Tris-HCl, 200 mM NaCl, 100 μM phenylmethylsulfonyl fluoride (PMSF), pH 8.0. Various stocks at 1, 5 and 20 mg ml⁻¹ were first kept at 4° C. for several weeks and then stored at −20° C. In this way, the PMSF will be sufficiently inactivated at 4° C.

Plasminogen-Activation Assay and Factor XII Activation Assay.

Plasmin (Pls) activity was assayed as described¹⁶. Peptides and proteins that were tested for their stimulatory ability were regularly used at 100 μg ml⁻¹. The tPA and plasminogen (Plg) concentrations were 200 μM and 1.1 μM, respectively, unless stated differently. Chromogenic substrate S-2251 (Chromogenix, Instrumentation Laboratory SpA, Milano, Italy) was used to measure Pls activity. Conversion of zymogen factor XII (#233490, Calbiochem, EMD Biosciences, Inc., San Diego, Calif.) to proteolytically active factor XII (factor XIIa) was assayed by measurement of the conversion of chromogenic substrate Chromozym-PK (Roche Diagnostics, Almere, The Netherlands) by kallikrein. Chromozym-PK was used at a concentration of 0.3 mM. Factor XII, human plasma prekallikrein (#529583, Calbiochem) and human plasma cofactor high-molecular weight kininogen (#422686, Calbiochem) were used at concentrations of 1 μg ml⁻¹. The assay buffer contained HBS (10 mM HEPES, 4 mM KCl, 137 mM NaCl, 5 μM ZnCl₂, 0.1% m/v albumin (A7906, Sigma, St. Louis, Mo., USA), pH 7.2). Assays were performed using microtiter plates (Costar, Cambridge, Mass., USA). Peptides and proteins were tested for their ability to activate factor XII. 150 μg ml⁻¹ kaolin, an established activator of factor XII was used as positive control and solvent (H₂O) as negative control. The conversion of Chromozym-PK was recorded kinetically at 37° C. for at least 60 minutes. Assays were done in duplicate. In control wells factor XII was omitted from the assay solutions and no conversion of Chromozym-PK was detected. In some assays albumin was omitted from the reaction mixture.

Alternatively, chromogenic substrate S-2222 (Chromogenix) was used to follow the activity of factor XII itself. With S-2222, activation of factor XII in plasma was measured, using 60% v/v plasma, diluted with substrate and H₂O with or without potential cofactor. Furthermore, auto-activation of factor XII was measured by incubating 53 μg ml⁻¹ purified factor XII in 50 mM Tris-HCl buffer pH 7.5 with 1 mM EDTA and 0.001% v/v Triton-X100, with S-2222 and H₂O with or without potential cofactor.

Surface Plasmon Resonance Studies

Binding of cross-β structure containing peptides/proteins was studied using surface plasmon resonance technology with a Biacore 2000 apparatus (Biacore AB, Uppsala, Sweden). A standardized amine coupling procedure was used to couple proteins with F domains to a CM5 chip (Biacore AB, Uppsala, Sweden). First, the dextran surface of the chips was activated by a 35 μl injection with a 1:1 mixture of 0.1 M N-hydroxysuccinimide (NHS) and 0.4 M N-ethyl-N′-(dimethylaminopropyl)carbodiimide (EDC) at a flow rate of 5 μl min.⁻¹. Then, the proteins were covalently coupled to the activated dextran surface. Remaining activated groups in each of the four flow channels were blocked by injection of 35 μl of 1 M ethanolamine hydrochloride pH 8.5. EDC, NHS and ethanolamine hydrochloride were obtained from Biacore. On one chip, on channels 1 to 4, buffer (reference channel), the soluble extracellular part of receptor for advanced glycation endproducts (sRAGE), tPA and K2P-tPA were immobilized. The immobilization buffer for the reference channel, channel 2 (sRAGE), channel 3 (tPA) and channel 4 (K2P-tPA) was 10 mM acetate pH 3.75. In channel 2, 2000 response units (RU) sRAGE was immobilized, 2700 RU and 2400 RU tPA and K2P-tPA are immobilized, respectively. The flow rate was 10 μl min.⁻¹, the injection time was 120″. The running buffer during immobilization was 10 mM HEPES pH 7.4, 140 mM NaCl. Buffers were filtrated on a 0.22 μm filter (white GSWP, 47 mm, Millipore) and degassed at room temperature. For subsequent binding experiments, the running buffer was 10 mM HEPES pH 7.4, 140 mM NaCl, 1.5 mM CaCl₂, 10 mM εACA, 0.005% Tween-20. Binding of albumin-AGE was determined with a solution of 3.9 μg ml⁻¹ albumin-AGE in running buffer. albumin-AGE was filtered on a Millex-GV 0.22 μm filter unit (Millipore). Binding of filtered Hb-AGE was tested at 32 μg ml⁻¹. Binding of amyloid γ-globulins were tested at 62.5 μg ml⁻¹. After each injection of protein, the chip was regenerated with 0.1 M H₃PO₄ pH 1.0. After injections with albumin-AGE and Hb-AGE this regeneration step was successful and sufficient, after injection with amyloid γ-globulins, the bound protein could not be released, not even after injection with more harsh regeneration buffers (HCl, NaOH). Binding of Hb-AGE was also tested after centrifugation for 10 min. at 16,000*g alternative to filtration. tPA activation before and after filtration was assessed with a Plg-activation assay. Also amyloid γ-globulins and amyloid endostatin (EntreMed, Inc., Rockville, Md., USA) were tested before and after centrifugation.

On a second chip, buffer, chemically synthesized HGFA F domain, chemically synthesized tPA F domain and Fn F4-5-His6, expressed in S. cerevisiae, were immobilized. HGFA F was immobilized in 10 mM acetate buffer pH 4.0, 190 RU. tPA F was immobilized in 5 mM maleate pH 5.5, 395 RU, Fn F4-5 in 5 mM maleate pH 6.0, 1080 RU. Now, the running buffer was 10 mM HEPES pH 7.4, 140 mM NaCl, 1.5 mM CaCl₂, 10 mM εACA, 0.05% Tween-20.

Regeneration buffer was running buffer supplemented with 1 M NaCl. Binding was tested with endostatin at 0-800 nM, Hb-AGE at 0-25 nM, recombinant β2-glycoprotein I (β2GPI) at 0-300 nM and 25 nM native Hb. For the Fn F4-5 channel, the maximum binding expressed in RU was plotted against the concentrations.

For both chips, channel 1 was used for reference purposes. The signal obtained with this channel was subtracted from the signals obtained with the channels with immobilized proteins.

Thioflavin T Fluorescence

Fluorescence of ThT—protein/peptide adducts was measured as follows. Solutions of 25 μg ml⁻¹ of protein or peptide preparations were prepared in 50 mM glycine buffer pH 9.0 with 25 μM ThT. Fluorescence was measured at 485 nm upon excitation at 435 nm. Background signals from buffer, buffer with ThT and protein/peptide solution without ThT were subtracted from corresponding measurements with protein solution incubated with ThT. Regularly, fluorescence of Aβ was used as a positive control, and fluorescence of FP10, a non-amyloid fibrin fragment¹⁶, was used as a negative control. Fluorescence was measured in triplicate on a Hitachi F-4500 fluorescence spectrophotometer (Ltd., Tokyo, Japan).

Congo Red Fluorescence

Solutions of 25 μg ml⁻¹ protein/peptide were incubated with 25 μM Congo red in PBS and fluorescence was measured at 590 nm upon excitation at 550 nm. Background signals from buffer, buffer with Congo red and protein/peptide solution without Congo red were subtracted from corresponding measurements with protein solution incubated with Congo red. Fluorescence was measured in triplicate on a Hitachi F-4500 fluorescence spectrophotometer (Ltd., Tokyo, Japan).

Transmission Electron Microscopy Imaging

For TEM analysis of protein en peptide solutions grids were prepared according to standard procedures. Samples were applied to 100-mesh copper grids with carbon coated Formvar (Merck, Germany), and subsequently washed with PBS and H₂O. Grids were applied to droplets of 2% (m/v) methylcellulose with 0.4% (m/v) uranylacetate pH 4. After a 2′-minutes incubation grids were dried on a filter. Micrographs were recorded at 80 kV, at suitable magnifications on a JEM-1200EX electron microscope (JEOL, Japan).

Structural Analysis of Formulated Protein Therapeutics

Formulated protein therapeutics were obtained from the local hospital pharmacy and were used as supplied by the manufacturers. The following protein therapeutics were purchased: 1) human growth hormone (GH) (Genotropin, batch 52344B51, 5 mg ml⁻¹ KabiQuick, Pharmacia B.V., Woerden, The Netherlands), 2) recombinant human Zn²⁺-chelated insulin (Monotard, batch NS61694, 100 IE ml⁻¹, Novo Nordisk, Bagsvaerd, Denmark), 3) human albumin (Cealb, batch NS61694, 200 mg ml⁻¹, Sanquin-CLB, Amsterdam, The Netherlands), 4) human modified gelatin (Gelofusine, batch 030606H4, 40 mg ml⁻¹, Braun Medical BV, Oss, The Netherlands), 5) rapid acting human insulin analogue (NovoRapid Flexpen, batch PH70008, 10 U ml⁻¹, Novo Nordisk), 6) blood cell growth factor filgrastim (Neupogen Singleject, batch N0693AD, 960 μg ml⁻¹, Amgen Europe, Breda, The Netherlands), 7) human-murine chimeric monoclonal antibody (Remicade-infliximab, batch 03D06H120A, 10 mg ml⁻¹, Centocor, Leiden, The Netherlands), 8) abciximab, an inhibitor of blood platelet aggregation (ReoPro, 2 mg ml⁻¹, Centocor, Leiden, The Netherlands) and 9) human coagulation factor VIII (FVIII) isolated from healthy volunteers (Aafact, lot 02L046250A, 3.6 mg ml⁻¹, Sanquin-CLB, Amsterdam, The Netherlands). Lyophilized therapeutics were dissolved according to the manufacturers recommendations. GH, zinc-insulin, Cealb and gelatin were stored at −20, 4, room temperature, 37 and 65° C. Other protein therapeutics were only kept at 4° C., and assayed for the presence of cross-β structure at shown time points. Enhancement in fluorescence of ThT and Congo red was measured with all formulated protein therapeutics. For this purpose, proteins were diluted to the indicated concentrations. In addition, tPA binding to the protein therapeutics was analyzed by ELISA and activation of tPA was tested using the Plg-activation assay. Zinc-insulin was diluted tenfold in the activation assay, GH was diluted to a final concentration of 500 μg ml⁻¹. Activation of factor XII and prekallikrein by the therapeutics was tested in the chromogenic factor XII assay (see above). For tPA ELISAs, 5 μg ml⁻¹ of the protein therapeutics were coated onto Greiner high-binding Microlon plates (#655092, Greiner Bio-One, Alphen a/d Rijn, The Netherlands). After coating, plates were blocked with Blocking Reagent (Roche Diagnostics, Almere, The Netherlands). A concentration series of tPA or K2P-tPA in PBS with 0.1% v/v Tween-20 and 10 mM ε-amino caproic acid was applied and the plates were incubated for 1 h at room temperature with constant swirling. Binding of tPA was assessed with monoclonal antibody 374b that binds to the protease domain of both tPA and K2P-tPA (American Diagnostica, Tebu-Bio, The Netherlands), peroxidase-conjugated rabbit anti-mouse immunoglobulins (RAMPO, P0260, DAKOCytomation, Glostrup, Denmark), and stained with 3′3′5′5′-tetramethylbezidine (TMB, catalogue number 4501103, buffer, catalogue number 4501401, Biosource Int., Camarillo, Calif., USA).

Activation of tPA by β₂-Glycoprotein I, Binding of Factor XII and tPA to β₂-Glycoprotein I, and ThT and TEM Analysis of β₂-Glycoprotein I

Purification of β₂-glycoprotein I (β₂GPI) was performed according to established methods^{20,21}. Recombinant human β₂GPI was made using insect cells and purified as described²⁰. Plasma derived β₂GPI as used in a factor XII ELISA, the chromogenic Plg-activation assay, was purified from fresh human plasma as described²¹. Alternatively, β₂GPI was purified from, either fresh human plasma, or frozen plasma (−20° C.) on an anti-β₂GPI antibody affinity column²².

Activation of tPA (Actilyse, Boehringer-Ingelheim) by β₂GPI preparations was tested in the Plg-activation assay (see above). Hundred μg ml⁻¹ plasma β₂GPI or recombinant β₂GPI were tested for their stimulatory cofactor activity in the tPA-mediated conversion of Plg to Pls, and were compared to the stimulatory activity of peptide FP13¹⁶.

Binding of purified human factor XII from plasma (Calbiochem) or of purified recombinant human tPA to β₂GPI purified from human plasma, or to recombinant human β₂GPI was tested in an ELISA. Ten μg of factor XII or tPA in PBS was coated onto wells of a Costar 2595 ELISA plate (Cambridge, USA) and incubated with concentration series of the two β₂GPI preparations. Binding of β₂GPI was assessed with monoclonal antibody 2B2²².

Binding of factor XII to β₂GPI was also tested using immunoblotting. β₂GPI (33 μg) purified either from fresh plasma or from frozen plasma was brought onto a 7.5% SDS-PAGE gel. After blotting to a nitrocellulose membrane, the blot was incubated with 1000× diluted rabbit polyclonal anti-human factor XII antibody (#233504, Calbiochem) and after washing with 3000× diluted peroxidase-conjugated swine anti-rabbit immunoglobulins (SWARPO, #P0399, DAKOCytomation, Glostrup, Denmark).

ThT fluorescence of β₂GPI was measured as follows. Purified β₂GPI from human plasma (400 μg ml⁻¹ final concentration) was incubated with or without 100 μM cardiolipin (CL) vesicles or 250 μg ml⁻¹ of the factor XII activator dextran sulphate 500k (DXS500k, Pharmacia, Uppsala, Sweden), in 25 mM Tris-HCl, 150 mM NaCl, pH 7.3. CL vesicles were prepared according to an established procedure. Briefly, CL was dried under a stream of nitrogen. The lipids were resuspended to a concentration of 10 mg ml⁻¹ in 25 mM Tris-HCl, pH 7.3, 150 mM NaCl by vigorous agitation, using a vortex. In the ThT fluorescence assay, fluorescence of β₂GPI in buffer, of CL or DXS500k in buffer, of buffer and ThT alone, and of β₂GPI-CL adducts and β₂GPI-DXS500k adducts, with or without ThT, was recorded as described above (section ThT fluorescence). In addition, TEM images were recorded with CL, β₂GPI from human plasma, with or without CL, and with recombinant β₂GPI, as described⁵.

Preparation of Amyloid-Like Ovalbumin, Human Glucagon, Etanercept and Murine Serum Albumin

To prepare structurally altered ovalbumin (OVA) with amyloid cross-β structures, purified OVA (Sigma, A-7641, lot 071k7094) was heated to 85° C. One mg ml⁻¹ OVA in 67 mM NaPi buffer pH 7.0, 100 mM NaCl, was heated for two cycles in PCR cups in a PTC-200 thermal cycler (MJ Research, Inc., Waltham, Mass., USA). In each cycle, OVA was heated from 30 to 85° C. at a rate of 5° C./min. Native OVA (nOVA) and heat-denatured OVA (dOVA) were tested in the ThT fluorescence assay and in the Plg-activation assay. In the fluorescence assay and in the Plg-activation assay, 25 and 100 μg ml⁻¹ nOVA and dOVA were tested, respectively. TEM images of nOVA and dOVA were taken to check for the presence of large aggregates.

Modified murine serum albumin (MSA) was obtained by reducing and alkylation. MSA (#126674, Calbiochem) was dissolved in 8 M urea, 100 mM Tris-HCl pH 8.2, at 10 mg ml⁻¹ final concentration. Dithiothreitol (DTT) was added to a final concentration of 10 mM. Air was replaced by N₂ and the solution was incubated for 2 h at room temperature. Then, the solution was transferred to ice and iodoacetamide was added from a 1 M stock to a final concentration of 20 mM. After a 15 min. incubation on ice, reduced-alkylated MSA (alkyl-MSA) was diluted to 1 mg ml⁻¹ by adding H₂O. Alkyl-MSA was dialyzed against H₂O before use. Native MSA (nMSA) and alkyl-MSA were tested in the ThT fluorescence assay and in the Plg-activation assay. In the ThT-fluorescence assay 25 μg ml⁻¹ nMSA and alkyl-MSA were tested, and in the Plg-activation assay 100 μg ml⁻¹ was tested. The presence of aggregates or fibrils was analyzed using TEM.

Amyloid-like properties in human glucagon (Glucagen, #PW60126, Novo Nordisk, Copenhagen, Denmark) were introduced using a modified protocol based on the method described by Onoue et al.²³. Lyophilized sterile glucagon was dissolved at 1 mg ml⁻¹ in H₂O with 10 mM HCl. The solution was subsequently kept at 37° C. for 24 h, at 4° C. for 14 days and again at 37° C. for 9 days. ThT fluorescence was determined as described above, and compared with freshly dissolved glucagon. tPA-activating properties of both heat-denatured glucagon and freshly dissolved glucagon was tested at 50 μg ml⁻¹. TEM analysis was performed to assess the presence of large multimeric structures.

### Example 1

Protein Assemblies with Cross-β Structure Bind to Immobilized Fibronectin Tvpe I Domains in a Biacore Surface Plasmon Resonance Set-Up

We used a surface plasmon resonance set-up of Biacore to test whether immobilized proteins with affinity for cross-β structure can capture amyloid-like polypeptides from solution under flow. This set up also allows to test suitable elution buffers to disrupt the interaction. In this way insight into suitable methods to deplete proteins with cross-β structures from solutions is obtained, as well as insight into how to compete for the interaction of cross-β structure binders, which are for example immobilized on beads in a column, with proteins comprising cross-β structures.

On one chip we immobilized sRAGE, tPA and K2P-tPA. One channel was left empty for reference purposes. Protein solutions were centrifuged for 10′ at 16,000*g before the solutions were applied to the Biacore chip. Centrifugation had no effect on the stimulatory effect of Hb-AGE and amyloid γ-globulins on tPA-mediated activation of Plg (FIG. 1A). Moreover, we filtrated all protein solutions before they were applied to the Biacore to exclude the presence of large aggregates with a density equal to buffer. For Hb-AGE similar response units were obtained after centrifugation or filtration (not shown). Subsequent experiments showed that Hb-AGE, albumin-AGE and amyloid γ-globulins bind to immobilized tPA and sRAGE (FIG. 1B-D). The interaction of tPA and sRAGE with Hb-AGE and albumin-AGE could be disrupted with 0.1 M H₃PO₄ buffer pH 1.0. Amyloid γ-globulins, however, were not removed by this buffer. After trying several more harsh regeneration buffers, the binding capacity of the chip was lost.

One a second chip, chemically synthesized HGFA F and tPA F, and Fn F4-5-His expressed in S. cerevisiae were immobilized. None of the polypeptides with cross-β structures bound to the two single F domain constructs. Hb-AGE, endostatin and recombinant β₂GPI bound, however, to the Fn F4-5 doublet, whereas native Hb did hardly bind (FIG. 1E-H). Affinities of the three proteins for Fn F4-5, expressed as the concentration of ligand that results in half maximum binding, ranges from 8 nM for Hb-AGE, via 165 nM for recombinant β₂GPI to up to 800 nM for endostatin. In fact, based on the absence of tPA activating properties in 100 μg ml⁻¹ endostatin (FIG. 1A), we did not expect any binding at all. Putatively, the surface plasmon resonance is more sensitive for the cross-β structure under the conditions used. We observed that when a stock solution of endostatin at 7.9 mg ml⁻¹ in the buffer as supplied by the manufacturer, is kept at ice or at room temperature, readily aggregates.

Perhaps, during the course of our experiments, part of the endostatin molecules start to denature, giving rise to the observed binding to Fn F4-5.

With this chip, interaction between Fn F4-5 and the protein ligands could be abolished simply by increasing the NaCl concentration from 140 mM to 1 M. This shows that the interaction was primarily based on charge interactions. Our surface plasmon resonance data show that F domains expressed in S. cerevisiae can bind to polypeptides with a cross-β structure. Furthermore, the data show that both 0.1 M H₃PO₄ buffer pH 1.0 and 10 mM HEPES pH 7.4, 1 M NaCl, 1.5 mM CaCl₂, 10 mM εACA, 0.05% Tween-20 are suitable buffers to release polypeptides with a cross-β structure from cross-β structure binding compounds. These buffers are also suitable to release cross-β structure binding compounds and proteins that are bound to a ligand with a cross-β structure. These data are helpful during the design of a method to deplete solutions from cross-β structure rich compounds by using cross-β structure binding polypeptides that are immobilized on a suitable supporting material.

### Example 2

Protein Solutions Contain Protein Aggregates with Cross-β Structure.

Structural Analysis of Proteins in Solution

We analyzed a series of protein solutions that are used as therapeutics for human use for the presence of cross-β structures in said protein. Protein solutions were stored at −20° C., 4° C. (as recommended by the manufacturers), room temperature, 37° C. or 65° C. Fluorescence of Congo red and ThT in the presence or absence of the proteins was analyzed, as well as tPA binding, tPA activation and factor XII activation. For fluorescence assays, 10 μg ml⁻¹ Aβ(1-40) E22Q amyloid was used as a positive control and gave typical values of approximately 1250 and 1800 A.U., respectively in the Congo red- and ThT fluorescence assay. Furthermore, TEM images were recorded to get insight whether amorphous aggregates are formed or fibrillar like structures. Gelatin, Cealb, FVIII and to some extent GH, stored at the recommended storage temperature of 4° C., enhanced the fluorescence of Congo red (FIG. 2A). In addition, Cealb, GH and FVIII enhance fluorescence of ThT (FIG. 2B). GH also induced tPA activation (FIG. 2C). Insulin activated tPA to a lesser extent, but still significantly (FIG. 2C). Both insulin and zinc-chelated insulin activate the factor XII/prekallikrein contact system (FIG. 2D). Gelatinous collagen fragments stored at 4° C. and 37° C. displayed enhanced Congo red fluorescence in a storage temperature dependent manner (FIG. 2E). Only gelatin kept at 37° C. activated factor XII (FIG. 2F). In an ELISA set-up, binding of tPA was established for Cealb, Reopro, gelatin, zinc-chelated insulin (FIG. 2G) and GH (FIG. 2H), all stored at the recommended temperature of 4° C. For both ELISAs, Hb-AGE was coated as a positive control (not shown for clarity). In the ELISA depicted in FIG. 2G, truncated K2P-tPA, or reteplase, which lacks the amyloid-binding F domain, was also tested for binding to the immobilized protein therapeutics. K2P-tPA did not bind to any of the therapeutics tested (not shown). On TEM images various condensed aggregates are seen with modified gelatin (FIG. 2I). GH appeared on TEM images as linear, branched and condense particles, all apparently composed of spherical particles (FIG. 2J). Zinc-chelated insulin appears on TEM images as thin linear unbranched fibrils with varying length (FIG. 2K). FVIII and Reopro did not appear as visible particles under the electron microscope. Cealb and insulin appeared as visible aggregates with no sign of a fibrillar nature (FIG. 2L, M). Reopro displayed storage temperature dependent ThT fluorescence enhancement properties and tPA activating properties (FIG. 2N, O). Only after storage at 65° C. ReoPro enhanced ThT fluorescence and induced Pls activity. Apparently, only at 65° C. ReoPro adopts the amyloid-like cross-β structure conformation. A TEM image of ReoPro that was stored at the recommended temperature of 4° C. revealed that some non-fibrillar aggregates were present, that apparently do not have ThT fluorescence enhancing or tPA activating properties under the conditions tested.

Protein Solutions Display Amyloid-Like Characteristics

Based on the observed binding of Congo red, ThT and tPA, based on the appearance on TEM images, and based on the observed activating properties towards tPA and factor XII, the tested solutions of Cealb, gelatin, insulin, zinc-insulin, GH, Reopro and FVIII displayed amyloid-like properties, when stored under recommended conditions. For Cealb, binding of tPA, Congo red and ThT is indicative for the presence of a cross-β structure. Binding of Congo red and activation of factor XII indicate the presence of cross-β structure conformation in gelatin. Binding of ThT and tPA, and activation of tPA by GH are indicative for amyloid-like properties in this solution. Finally, both activation of tPA and factor XII by insulin/zinc-insulin are indicative for the presence of cross-β structure rich aggregates. These data show the presence of protein or peptide aggregates with amyloid-like properties or the potential that the cross-β structure can be formed upon storage in these protein solutions.

Structural analysis of the tested proteins can be expanded using techniques and assays such as X-ray diffraction experiments, Fourier transform infrared spectroscopy, size exclusion HPLC, CD spectropolarimetry and binding assays using amyloid binding proteins, and can be expanded by introducing new protein solutions in the series of analyses.

### Example 3

Structure Analysis of Various β₂-Glyvcorotein I Preparations

Factor XII and tPA Bind to Recombinant β₂GPI and to β₂GPI Purified from Frozen Plasma, but not to β₂GPI Purified from Fresh Plasma

Recombinant β₂GPI, but not β₂GPI purified from fresh plasma stimulate tPA-mediated conversion of Plg to Pls, as measured as the conversion of the Pls specific chromogenic substrate S-2251 (FIG. 3A). An ELISA demonstrated that tPA and factor XII bind recombinant β₂GPI, but not to β₂GPI purified from fresh human plasma (FIG. 3B, C). Recombinant β₂GPI binds to factor XII with a k_{D} of 20 nM (FIG. 3C) and to tPA with a k_{D} of 51 nM (FIG. 3B). In addition, factor XII co-elutes fiom the anti-β₂GPI antibody affinity column with β₂GPI, that was purified from plasma that was frozen at −20° C. and subsequently thawed, as shown on Western blot after incubation of the blot with anti-factor XII antibody (FIG. 3D). This shows that β₂GPI refolds into a conformation containing cross-β structures upon freezing. FIG. 3F shows that exposure of β₂GPI to CL or DXS500k introduces an increased ThT fluorescence signal, indicative for a conformational change in β₂GPI accompanied with the formation of cross-β structure conformation. Again, recombinant β₂GPI initially already gave a higher ThT fluorescence signal than native β₂GPI purified from plasma. These data not only show that recombinant β2GPI already comprises more cross-β structure conformation than plasma β₂GPI, but that recombinant β₂GPI also adopts more readily this conformation when environmental factors change. In FIG. 3G it is shown that exposure of β₂GPI to CL, immobilized on the wells of an ELISA plate, renders β₂GPI with tPA binding capacity. Binding of β₂GPI directly to the ELISA plate results in less tPA binding. These observations also show that CL has a denaturing effect, thereby inducing amyloid-like conformation in β₂GPI, necessary for tPA binding. These observations, together with the observation that exposure of β₂GPI to CL vesicles induced ThT binding capacity (FIG. 3F), show that exposure of β₂GPI to a denaturing surface induces formation of amyloid-like cross-β structure conformation. Furthermore, large fibrillar structures are seen on TEM images of plasma β₂GPI in contact with CL (FIG. 3G, image 2 and 3). Small CL vesicles seem to be attached to the fibrillar β2GPI. Images of plasma β₂GPI alone (FIG. 3G, image 1) or CL alone (not shown) revealed that no visible ultrastructures are present. In contrast, non-fibrillar aggregates and relatively thin curly fibrils can be seen on images of recombinant β₂GPI (FIG. 7G, image 4). These observation show that exposure of β₂GPI to CL and expression and purification of recombinant β₂GPI result in an altered multimeric structure of β₂GPI, when compared to the monomeric structure observed with X-ray crystallography²⁴. Exposure of β₂GPI to CL or DXS500k induces an increased fluorescence when ThT is added, indicative for the formation of cross-β structure conformation when β₂GPI contacts a negatively charged surface. We predict that the cross-β structure can be relatively easily formed by one or more of the five domains of the extended β₂GPI molecule²⁴. Each domain comprises at least one β-sheet that may function as a seed for local refolding into a cross-β structure.

In conclusion, it is revealed that several physical/chemical conditions influence the fold of the protein. Exposure to CL or DXS500, a freeze-thaw cycle, variations in protein purification protocol, the source of the protein and exposure to plastic all introduce a structural rearrangement in the protein accompanied by the formation of the amyloid-like cross-β structure fold. This new fold can be detected by, amongst others, tPA binding, tPA activation, factor XII binding and by conventional amyloid fluorescence assays.

### Example 4

Induction of Cross-β Structures in Proteins

OVA with amyoid-like properties was obtained by heat denaturation at 85° C. (FIG. 4A, B, I, K). The presence of cross-β structures was established with ThT fluorescence and Plg-activation assays and by TEM imaging. The fibrillar structures of at least up to 2 μm in length, seen on the TEM images are likely not the only OVA assemblies with cross-β structures present, as concluded from the observation that filtration through a 0.2 μm filter does not reduce the enhancement of ThT fluorescence. A person skilled in the art can perform similar experiments with murine serum albumin (MSA), human glucagon or Etanercept, such as those described below (FIG. 4).

The amyloid-like protein fold was induced in MSA by heat denaturation at 85° C. and by reduction and alkylation of disulphide bonds (FIG. 4A-D). We observed that also native MSA enhanced ThT fluorescence to some extent, but this was not reflected by stimulation of tPA activation. Although heat-denatured MSA and alkylated MSA enhance ThT fluorescence to a similar extent, they differ in tPA activating potential. This suggests that tPA and ThT interact with distinct aspects of the cross-β structure. Previously, we observed that Congo red, another amyloid-specific dye, can efficiently compete for tPA binding to amyloid-like aggregates in ELISAs, whereas ThT did not inhibit tPA binding at all (patent application P57716EP00 and B.Bouma, unpublished data).

Amyloid-like cross-β structure conformation was induced in glucagon by heat-denaturation at 37° C. at low pH in HCl buffer (FIG. 4E, F, J). In this way, a potent activator of tPA was obtained, that enhanced ThT fluorescence to a large extent. In addition, long and bended unbranched fibrils were formed, as visualized on TEM images (FIG. 4J). Noteworthy, at high glucagon concentration, also native glucagon had some tPA activating potential, indicative for the presence of a certain amount of cross-β structure rich protein.

Alkylated Etanercept did not activate tPA at all, whereas heat-denatured Etanercept had similar tPA activating potential as amyloid γ-globulins (FIG. 4G). After heat denaturation, Etanercept also efficiently induced enhanced ThT fluorescence (FIG. 4H). Native Etanercept both induced some tPA activation and gave some ThT fluorescence enhancement.

From our analyses we conclude that dOVA, alkyl-MSA, heat/acid-denatured glucagon and heat-denatured Etanercept comprise the cross-β structure conformation. The presence of the cross-β structures can be further established by circular dichroism spectropolarimetry analyzes, X-ray fiber diffraction experiments, Fourier transform infrared spectroscopy, Congo red fluorescence/birefringence, tPA binding, factor XII activation and binding, and more.

### Example 5

Introduction

The following examples show that with proteins or protein fragments with affinity for amyloid-like misfolded proteins, affinity matrices can be constructed that specifically extract misfolded protein from buffer or complex protein solutions, thereby depleting the protein solutions from potentially harmful cytotoxic or immunogenic or functionally hampered or otherwise unwanted obsolete molecules. In addition to (recombinant) protein (fragments) or as an alternative to such protein (fragments), small molecules with affinity for one or more misfolded proteins can be applied in detection and/or depletion applications. We provide here the use of ellagic acid in a misfolded protein depletion experiment. This extraction technology also allows for detection of misfolded protein, and furthermore, the technique facilitates subsequent identification of the misfolded protein. Moreover, the example demonstrates that in aqueous solutions, protein molecules, that have amyloid-like protein conformation, are identified with our technology.

Methods: Preparation of Misfolded Protein Affinity Matrix

Expression

Synthetic genes of human BiP, human fibronectin finger 4,5 (Fn F4,5) fragment, and human tissue-type plasminogen activator finger EGF (tPA F-EGF) fragment were ordered from Geneart (Regensburg, Germany). These DNA constructs were digested using BamHI and NotI, and ligated into vector pABC674 (ABC-expression facility, Utrecht University, The Netherlands), which contains a carboxy-terminal FLAG-tag—His-tag. HEK293E cells were transiently transfected with these constructs using the polyethylene-imine method, and grown for 5-6 days.

Purification

The cells were pelleted by centrifugation and the supernatant was concentrated on a Quixstand concentrator (A/G Technology corp.), using a 30 or 5 kDa cut-off filter (GE Healthcare) for BiP and for Fn F4,5 and tPA F-EGF, respectively. A dialysis step was performed on the same concentrator, and the proteins were dialysed either against PBS+0.85 M NaCl pH 7.4 (BiP), or against 25 mM Tris pH 8.2+0.5 M NaCl (Fn F4,5 and tPA F-EGF). The concentrated and dialysed medium was filtered (0.45 μm, Millipore) and incubated with Ni-Sepharose beads (GE-Healthcare, catalogue number 17-5318-02) in the presence of 10-20 mM imidazole, for either 3 h at room temperature or overnight at 4° C. under constant motion. A column was packed with the beads and the proteins were extracted by increasing imidazole concentration. The proteins purified in this way had a purity of 80-90%, as established by SDS-PAGE (Invitrogen, NuPage 4-12% BisTris NP0323), using MOPS buffer (Invitrogen NP0001) for BiP or MES buffer (Invitrogen NP0002) otherwise, and Coomassie stain (Fermentas PageBlue R0571).

Affinity Determination

Denatured proteins and their native controls BSA (Sigma, A7906), glycated BSA, Hb (Sigma, H7605), glycated Hb, ovalbumin (Sigma, A6741), heat-denatured misfolded ovalbumin, human γ-globulins (Sigma, G4386), heat-denatured misfolded γ-globulins, alkyl-γ-globulins, lysozyme (ICN Biochemicals, 100831) and alkyl-lysozyme were coated on ELISA plates (Greiner Microlon high-binding, 655092) in 50 mM NaHCO₃-buffer pH 9.6. The plates were blocked using Blocking reagent (Roche 1112589). A dilution series of the protein of interest was applied to the coated proteins and wells were subsequently washed using TBS-T (50 mM Tris pH 7.3, 150 mM NaCl and 0.1% Tween20). Bound protein is detected by the FLAG-tag using 1:3000 anti-FLAG-HRP (Sigma A-8592) in PBS-T, or by 1:1000 Ni-NTA-HRP (Qiagen 34530) in PBS-T. The HRP reaction Is performed using the TMB substrate (Biosource 4501103 or Tebu Bio 101TMB100-500), stopped using 10% H₂SO₄ and absorbance was measured at 450 nm.

Preparation, Expression and Purification of sRAGE-His

The soluble extra-cellular fragment of human receptor for advanced glycation end-products (sRAGE) was cloned, expressed and purified as follows (Q.-H. Zeng, Prof. P. Gros, Dept. of Crystal- & Structural Chemistry, Bijvoet Center for Biomolecular Research, Utrecht University, Utrecht, the Netherlands, and Cor Seinen, Department of Clinical Chemistry and Haematology, University Medical Center Utrecht, the Netherlands). Human cDNA of RAGE was purchased from RZPD (clone IRALp962E1737Q2, RZPD, Berlin, Germany). For PCRs, the gagatctGCTCAAAACATCACAGCCCGG forward primer was used comprising a BgIII site, and the gcggccgcCTCGCCTGGTTCGATGATGC reverse primer with a NotI site. The soluble extracellular part of RAGE comprises three domains spanning amino-acid residues 23-325. The PCR product was cloned into a pTT3 vector, containing an amino- or carboxy-terminal His-tag. The sRAGE was expressed in 293E hamster embryonic kidney cells at the ABC-protein expression facility (Utrecht University, Utrecht, The Netherlands). Concentrated cell culture medium was applied to a Hi-trap Chelating HP Ni²⁺-NTA column (Amersham Biosciences Europe, Roosendaal, The Netherlands). The running buffer was 25 mM Tris-HCl, 500 mM NaCl, pH 8.0. The protein was eluted by using a step gradient of 0 to 500 mM imidazole. Purity of the His-sRAGE was depicted from Coomassie stained SDS-PAGE gels. After concentration, the buffer was exchanged to 20 mM Tris-HCl, 200 mM NaCl, pH 8.0.

Matrix Preparation

After His-tag based purification, pooled fractions with construct were dialysed in a 3.5 kDa cut-off membrane (Spectra/Por 132720) against their column running buffer without imidazole. Occasionally occurring precipitates were removed by centrifugation (30 minutes at 16.000*g) or filtration (0.45 μm). Ni-Sepharose beads (GE-Healthcare17-5318-02) were incubated overnight with dialysed protein at 4° C. in the presence of 20 mM imidazole. After discarding the protein solution, beads were washed 5× using PBS+0.1% Tween20+20 mM imidazole (PBS-TI).

Depletion Experiments

BSA-AGE was diluted in PBS-TI to a concentration of 50 μg/ml. This solution was ultra-centrifuged at 100,000*g for 1 hour at 4° C. The resulting solution had a concentration of approximately 45 μg/ml. This was diluted 10-fold for the fishing experiments (working concentration: 4.5 μg/ml). Fishing experiments were performed in PBS-TI, 256-fold diluted human serum in PBS-TI or 512-fold diluted human plasma in PBS-TI, with or without 4.5 μg/ml BSA-AGE. Forty μl 50% beads suspension in PBS-TI was added to 170 μl solution with or without BSA-AGE and incubated overnight at 4° C. under constant motion. Unbound material was extracted and tested for BSA-AGE content in a sandwich ELISA set-up.

BSA-AGE Detection by Sandwich ELISA

For BSA-AGE sandwich ELISA, anti-AGE monoclonal antibody 4B5 (⁵) was coated to an ELISA plate (Greiner Microlon high-binding, 655092), which was subsequently blocked. Solutions containing BSA-AGE were allowed to bind for 1 h at room temperature. BSA-AGE was detected using polyclonal rabbit anti-HSA (DakoCytomation, A0001; 1:1000 in PBS with 0.1% Tween20 (PBS-T)) followed by SWARPO (DakoCytomation, P0217; 1:4000 in PBS-T). The peroxidase reaction was performed using 100 μl of OPD in phosphate citrate buffer pH 5, and stopped by 50 μl of 10% H₂SO₄ and measured at 490 nm.

Platelet Aggregation Assay with Protein Solutions Depleted of Amyloid-Like Protein

I. Coupling of tPA F-Biotin to Streptavidin-Sepharose

Total chemically synthesized lyophilized tPA F-biotin (T.Hackeng,University Maastricht) was dissolved at 5 mg/ml in 20 mM HEPES, 137 mM NaCl, 4 mM KCl, pH 7.4; HBS). For preparation of affinity matrix 175 μl Streptavidin-Sepharose (Amersham Biosciences AB, SE-751 84 Uppsala Sweden, 17-5113-01) was washed 10 times with 175 μl 1× HBS. Filter tubes (Millipore Non-Sterile Ultrafree MC 5 μM filter unit, UFC30SV00 Millipore Corporation Bedford Mass. 01730 USA) were used to wash beads. An Eppendorf table-top centrifuge was used, 30 seconds at 500 rcf. Hundred-twenty μl tPA F-biotin was added to beads depleted from buffer by centrifugtion. Approximately 0.6 mg tPA F-biotin was added to beads. Coupling procedure was according to the guidelines of the manufacturer. Incubation of beads and tPA F-biotin was done under constant motion at room temperature for 1 hour. Beads were subsequently washed 12×. Wash buffer was analyzed for tPA F-biotin content to allow for determination of the coupling efficiency. Beads with bound tPA F-biotin awee stored in HBS at 4° C. The coupling procedure was performed in parallel with control beads, omitting the tPA F-biotin. Coupling efficiency was assessed using ELISA. A concentration series of tPA F-biotin was immobilized on the well of a 96-wells plate (Greiner Microlon high-binding). The tPA F-biotin solution after contacting the Streptavidin-Sepharose beads, as well as the wash buffer after washing tPA F-biotin contacted Streptavidin-Sepharose was diluted in coat buffer, accordingly, and also coated. The plate was blocked with Blocking Reagent (cat.no. 37545, Pierce, Perbio Science Nederland B. V., Etten-Leur, The Netherlands). Detection antibody used was Streptavidin-HRP, 1:1000 dilution (cat.no. P0397, Dako, Heverlee, Belgium). TMB substrate (100 μl/well) was used for staining (cat.no. 4501103, 4501401, Biosource, Invitrogen, Breda, The Netherlands) and the stain reaction was stopped with 50 μl of 10% H₂SO₄. Absorbance was measured at 450 nm. Incubations were for 30 minutes at room temperature under constant shaking. Washes (5× times between incubation steps) and dilutions werre in PBS with 0.1% Tween20.

II. Depletion of Amyloid-Like Misfolded Protein from Protein Solutions

A spike of 1 μg/ml ultracentrifuged BSA-AGE was added to PBS/0.1% v/v Tween20 or to 512-fold diluted single human donor plasma in PBS/0.1% v/v Tween20. Solutions were added to either control beads or to tPA F-biotin Streptavidin-Sepharose. The solution after incubations was analyzed for the presence of remaining BSA-AGE, in a sandwich ELISA, as described above.

In a next series of experiments, diluted plasma was enriched with a 250 μg/ml BSA-AGE spike and subsequently added to tPA F-biotin—Streptavidin-Sepharose. After contacting 150 μl of the plasma with BSA-AGE spike for 2 hours under constant motion, to 15 μl of the affinity matrix for depletion of misfolded proteins, the supernatant was analyzed for its property to induce platelet activation resulting in their aggregation. Results are compared to platelet activating properties of the spiked plasma before depletion of BSA-AGE.

Freshly drawn human aspirin free blood was mixed gently with citrate buffer to avoid coagulation. Blood was spinned for 15′ at 150*g at 20° C. and supernatant was collected; platelet rich plasma (PRP) with an adjusted final platelet number of 200,000 platelets/μl. Platelets were kept at 37° C. for at least 30′, before use in the assays, to ensure that they were in the resting state. For the aggregometric assays, 270 μl platelet solution was added to a glass tube and prewarmed to 37° C. A stirring magnet was added and rotation was set to 900 rpm, and the apparatus (Whole-blood aggregometer, Chrono-log, Havertown, Pa., USA) was blanked. A final volume of 30 μl of tester solution was added, containing the agonist of interest (buffer, control, diluted plasma with BSA-AGE, before and after contacting tPA F-biotin—Streptavidin-Sepharose), prediluted in HEPES-Tyrode buffer pH 7.2. Aggregation was followed in time by measuring the absorbance of the solution, that will decrease in time upon platelet aggregation. As a positive control synthetic thrombin receptor activating peptide TRAP was used. Aggregation was recorded for 15′ and expressed as the percentage of the transmitted light (0-100%).

Materials and Methods: Binding of LRP to Misfolded Protein.

Cloning and Expression of LRP Cluster IV.

Cluster IV of the low-density lipoprotein (LDL) receptor-related protein (LRP cl-IV) was cloned from complete cDNA of THP1 cells by PCR using the following forward and reverse primers: GGATCCTCCAACTGCACGGCTAGC (oLRPIVF) and GCGGCCGCGATGCTGCAGTCCTCCTC (oLRPIVR) introducing BamHI and NotI sites (underlined), respectively at the amino- and carboxy-terminus of cluster IV. This PCR fragment was cloned into TOPO TA vector (Invitrogen). The sequence was verified and the construct was subsequently cloned in the pABC-based expression vector 675 (ABC-expression facility, Utrecht University, The Netherlands) using the BamHI and NotI sites. This vector introduces an amino-terminal cystatin signal sequence to the expressed protein of interest enabling secretion into the medium. Furthermore it has a carboxy-terminal FLAG-HIS tag for purification and detection purposes.

Two and a half μg of the obtained construct was transfected into 5 ml HEK293E/S cells, using the polyethylene-imine method, and medium was harvested after one week of cell culturing by centrifugation at maximum speed for 20 seconds (performed by the ABC-expression facility). Presence of expressed LRP cl-IV was verified by analyzing a Western blot after staining with anti-FLAG-tag antibody and chemiluminescent compound. The cell culture supernatant comprising LRP cluster IV protein was used directly without further purification for the ELISA experiments (see below).

Enzyme Linked Immunosorbent Assay for Testing of LRP Cluster IV Binding to Misfolded Proteins.

Binding of LRP cl-IV to misfolded protein was determined using an enzyme linked immuno sorbent assay (ELISA) set-up. For this purpose 50 μl of a 5 μg/ml solution of BSA, BSA-AGE, Hb or Hb-AGE or coat buffer (for negative control) was coated for 1 h with motion. Proteins were diluted in coat buffer (100 mM NaHCO₃ pH 9.6). The BSA and Hb controls were prepared freshly by dissolving proteins at 1 mg/ml in PBS by rolling for 10 minutes on a roller bank at room temperature, 10 minutes incubation at 37° C. followed by again 10 minutes incubation at the roller bank. Coat controls were performed with anti-glycated protein antibody 4B5, anti-albumin antibody or anti Hb antibody. After coating the plates were washed twice with PBS/0.1% Tween-20 (v/v) and blocked with 300 μl/well blocking reagent (Roche Diagnostics, Almere, The Netherlands) for 1 h at room temperature with motion. Plates were washed twice and incubated in duplicate with a dilution series of medium containing LRP cl-IV (5, 50 or 500 times diluted cell culture supernatant) in PBS/0.1% Tween-20 (v/v) or buffer control for 1 h at room temperature with motion. After five wash cycles, a HRP conjugated anti FLAG antibody or, for the coat controls, anti-glycated protein antibody, anti-albumin antibody or anti-Hb antibody, was added to the wells (50 μl). The anti-FLAG antibody was diluted 3000 times, the anti-glycated protein antibody, the anti-albumin antibody and the anti-Hb antibody were diluted 1000 times, all in PBS/0.1% Tween-20 (v/v). After five washes with wash buffer binding of antibody was assessed with a secondary antibody. For the coat controls, RAMPO (3000 times diluted) was used to monitor binding of anti-glycated protein antibody, SWARPO (3000 times diluted) was used to monitor binding of anti-albumin antibody and anti-Hb antibody. No secondary antibody was needed to monitor binding of anti-FLAG antibody since HRP is conjugated to this antibody. After 5 washes with wash buffer, binding of anti-FLAG antibody and secondary antibodies was assessed with 100 μl/well TMB substrate (ready to use from Tebu Bio). The reaction was stopped by adding 50 μl/well of 2 M H₂SO₄ in H₂O. After ˜2 minutes absorbance was read at 450 nm.

To test whether amyloid-like crossbeta structure binding compounds tPA, Congo red, Thioflavin T and Thioflavin S interfere with LRP cl-IV binding to BSA-AGE, concentration series of the potential inhibitory amyloid binding moieties were tested in the presence of 50 times diluted medium containing LRP cl-IV. The following inhibitors were used: tPA, Congo red, Thioflavin T (ThT) and Thioflavin S (ThS). As a control to tPA, K2P tPA, which lacks the amyloid-like misfolded protein binding finger domain, was included in the analyses. The influence of tPA and K2P tPA was tested in the presence of 10 mM ε-amino caproic acid to avoid binding of the kringle2 domain of tPA and K2P tPA to lysine- and arginine residues. Binding buffer and K2P tPA serve as negative controls in these inhibition studies. The concentration series was measured in triplicate, the values averaged and standard deviations calculated. Background signals obtained with buffer-coated wells were subtracted. Signals obtained with binding of LRP cluster IV to BSA-AGE was set arbitrarily to a reference binding of 100% and signals obtained with the concentration series of misfolded protein binding moieties and K2P tPA were calculated based on this set reference.

Materials & Methods: Detection of Misfolded β2-Glycoprotein I

Stock Solutions

Stock Solution of β2-Glycoprotein I; 800 μg/ml in 1× Tris Buffered Saline, pH 7.2 (1× TBS)

Cardiolipin vesicles were prepared from a lamellar solution of cardiolipin (Sigma; C-1649) according to a protocol by Subang et al. (²⁵). Twohundred μl of cardiolipin was placed into a glass tube and ethanol was evaporated by a constant stream of N₂. The dried cardiolipin was reconstituted in 104 μl of 1× TBS and vortexed thoroughly. The resulting solution contained 10 mg/mL (7.14 mM) of cardiolipin vesicles. This solution could be stored for 14 days at 4° C., maximally. All dilutions were in TBS and after storage, the solution was vortexed before use.

Modifications: Preparation of Alkyl-β2gpi

β2-GPI was reduced and alkylated as follows. Sixhundredforty μl of β2-GPI stock was mixed with 640 μl of 8 M Urea (cooled solution) in 0.1 M Tris pH-8.2. The solution was degassed with N₂ gas for approximately 6 minutes. From a 1 M DTT stock 12.8 μl was added to the solution, mixed and incubated for 3 hours at room temperature. A 1 M Iodoacetamide (Sigma; I-6125) was prepared, of which 25.6 μl was added to the β2-GPI reaction mixture. The solution was subsequently dialysed against PBS. Misfolding of the resulting alkyl-β2gpi was established by the enhancement of Thioflavin T fluorescence and by the increased ability to activate tPA/plasminogen, resulting in plasmin in the chromogenic assay. The chromogenic assay is performed with 400 μM tPA, 100 μg/ml plasminogen. Signals obtained with alkyl-β2gpi are compared with those obtained with native β2gpi starting material and with positive control acid/heat denatured amyloid-like misfolded γ-globulins.

Methods for Structural Analyses of Protein Solutions Used as Biopharmaceuticals

tPA binding assay with immobilized biopharmaceuticals in an ELISA Nunc Immobilizer plates (Nalge Nunc, #436013, Rochester, N.Y., USA) were coated with 50 μL containing 5 μg/mL of sample protein (unless indicated otherwise) in 100 mM NaHCO3, pH 9.6, 0.05% m/v NaN₃ for 1 hour at room temperature. Plates were washed twice with Tris buffered saline pH 7.2 containing 0.1% Tween20 (TBST) and blocked with PBS containing 1% Tween20 for 1 hour at room temperature. Plates were washed twice with TBST and incubated, in duplicate, with a concentration series of either tPA (Actilyse, Alteplase; Boehringer-Ingelheim, Alkmaar, The Netherlands) or a truncated form of tPA (Reteplase; Rapilysin, Roche Diagnostics GmbH, Mannheim Germany), lacking the amyloid binding domain, diluted in PBS containing 0.1% Tween 20 (PBST). We found that the finger domain interacts with amyloid-like misfolded proteins (unpublished data). Incubations were performed for 1 hour at room temperature in the presence of 10 mM ε-amino caproic acid (εACA). εACA is a lysine analogue and is used to avoid potential binding of tPA to lysine-containing ligands via its kringle2 domain. Plates were washed five times with TBST and incubated with antibody 374b α-tPA (American Diagnostica, Instrumentation Laboratory, Breda, The Netherlands) diluted 1:1000 in PBST for 1 hour at room temperature. Plates were washed five times with TBST and incubated with peroxidase labeled anti-mouse immunoglobulins (RAMPO; DAKOCytomation, Glostrup, Denmark) diluted 1:3000 in PBST for 30 minutes at room temperature. Plates were washed five times with PBS 0.1% Tween20, and stained with 100 μL/well of tetramethyl-benzidine (TMB) substrate (Biosource Europe, Nivelles, Belgium). The reaction was terminated with 50 μL/well of 2 M H₂SO₄ and substrate conversion was read at 450 nm on a Spectramax340 microplate reader. Curves were fitted with a one-site binding model (GraphPad Prism version 4.02 for Windows, Graphpad Software, Calif., USA) from which Kd and Bmax were determined.

tPA/Plasminogen Activation Assay

Exiqon Peptide Immobilizer plates were blocked for 1 hour with PBS, 1% Tween20 and rinsed twice with distilled water. The conversion of the chromogenic substrate S-2251 (Chromogenix, Italy) by plasmin was kinetically measured at 37° C. on a Spectramax340 microplate reader at a wavelength of 405 nm. The assay mixture contained 400 μM tPA, 100 μg/mL plasminogen (purified from human plasma) and 415 μM S-2251 in HEPES buffered saline (HBS) pH 7.4. Denatured γ-globulins (100 μg/ml) with amyloid-like structure was used as reference and positive control. Lyophilized γ-globulins (Sigma, Mo., USA) were dissolved in a 1(:)1 volume ratio of 1,1,1,3,3,3-hexafluoro-2-propanol and trifluoro-acetic acid and subsequently dried under air. Dried γ-globulins was dissolved in H₂O to a final concentration of 1 mg/ml and kept at room temperature for at least three days and subsequently stored at −20° C. Maximal tPA activating capacity was determined from the linear increase seen in each activation curve and expressed as a percentage of the standardized positive control. To confirm tPA dependence of plasmin generation, all samples were assayed for their ability to convert plasminogen into plasmin in absence of tPA.

Analyses of Protein Therapeutics

Protein therapeutics were obtained from the local hospital pharmacy and analyzed within the expiry limits as stated by the manufacturers. Five μL of the various protein therapeutics were tested for their ability to enhance both ThT and CR fluorescence. tPA activating capacity of the protein therapeutics was determined in 1:10 diluted samples (unless indicated otherwise). tPA binding ELISA's were performed by coating protein therapeutics 1:10 in 100 mM NaHCO₃, pH 9.6, 0.05% m/v NaN₃.

Stability Testing of Biopharmaceuticals

To mimic accelerated stability testing several therapeutics were exposed to denaturing conditions and assayed for amyloid-like properties before and after treatment by tPA activation assay at 100 μg/mL protein and ThT fluorescence enhancement assay at 25 μg/mL protein. For this purpose, 5 mg/mL Glucagon (Glucagen; Novo Nordisk Farma B. V., Alphen aan de Rijn, The Netherlands) was incubated at 37° C. in 0.01 M HCl for 48 hours. One mg/mL Etanercept (Enbrel; Wyeth Pharmaceuticals B. V., Hoofddorp, The Netherlands) in 67 mM sodium phosphate buffer, 100 mM NaCl pH 7.0 was gradually heated from 30° C. to 85° C. over a period of 12 minutes and afterwards cooled to 4° C. for 5 minutes, this treatment was repeated 4 times. Abciximab (Reopro; Centocor B. V., Leiden, The Netherlands) and Infliximab (Remicade; Schering-Plough B. V., Utrecht, The Netherlands) were incubated at 65° C. for 16 and 72 hours, respectively.

Analyses of the Misfolded Protein Binding Properties of Ellagic Acid: Incorporation of Ellagic Acid in Misfolded Protein Depletion Technology

Materials for screening of ellagic acid for its ability to interact with misfolded proteins
Microlon high binding plates, (Greiner) NR 655092
Blocking Reagent, (Roche)
tPA Actilyse, (Boehringer Ingelheim)
Anti-tPA 374B, (American Diagnostica) Prod. No. 374B
RAMPO, 1,3 g/L (DakoCytomation) product number P0260
TMB (TebuBio)
Congo red (Aldrich Chemicals, Germany), cat. Number 86,095-6
Thioflavin T (Sigma-Aldrich, Germany), cat number T-3516
Ellagic acid, 5 mg/ml in DMSO (TimTec, Newark, Del., USA, www.timtec.net)
ellagic acid hydrate (Sigma-Aldrich Chemie GmbH, Steinheim, Germany), catalogue number 37,274-9 (10 gr.)

ELISA: Binding of tPA to Glycated Haemoglobin, Heat-Denatured Misfolded Ovalbumin and Amyloid-β; Influence of Ellagic Acid

Aim:

Determination of the influence of ellagic acid on the binding of a suboptimal concentration of tPA to a refined concentration of immobilized Hb-AGE. Parameter: a fixed concentration of tPA, in PBS with 0.1% (v/v) Tween20, 10 mM εACA and 10% DMSO, mixed with ellagic acid. As a positive control, tPA in binding buffer with 10% DMSO was used. This binding was set to 100% and values obtained after co-incubation of Hb-AGE with tPA with ellagic acid, were scaled accordingly. Binding experiments were performed in single wells, in a duplicate experiment. Binding data obtained with this duplicate experiment was averaged. Enhancement of tPA binding was arbitrarily set to values>100%. Inhibition of tPA binding to misfolded Hb-AGE was arbitrarily set to values smaller than 50%. With the chosen experimental lay-out, it can not be distinguished whether ellagic acid influences the interaction between tPA and Hb-AGE by binding to Hb-AGE only or by binding to tPA only or by binding to both tPA and Hb-AGE.

In a next series of experiments, the ability of ellagic acid that interacts with tPA and/or Hb-AGE, to bind to immobilized fb-AGE, heat-denatured misfolded ovalbumin and amyloid-β was analyzed. In a first experiment, the coated misfolded Hb-AGE was first incubated with ellagic acid. After washing, tPA binding was assessed. Similar binding assays are performed with misfolded OVA and Aβ. Finally, concentration series of ellagic acid are used in the binding studies.

Experimental Procedure in Brief

1. Hb-AGE coat at 1.25 μg/ml in Coat buffer on a Greiner Microlon high-binding plate, for 30 minutes at room temperature, with agitation. Coat buffer in control wells.

2. wash three times with wash buffer

3. Block all wells with 300 μl 0.5*recommended concentration Roche blocking reagent for 30 minutes at room temperature, with agitation

4. wash twice

5. Stock tPA: 50 μM Actilyse
1) tPA incubation at 0.5 nM (with tenfold diluted ellagic acid stock of 5 mg/ml in DMSO)
2) tPA incubation at 0.5 nM tPA with 1 mM ThT (control) with 10% DMSO
3) tPA incubation at 0.5 nM tPA with 0.5 mM CR (control) with 10% DMSO

6. Incubate for 30 minutes at room temperature, with agitation

7. wash five times with TBS/0.1% Tween20

8. Fill wells with mouse monoclonal anti-tPA antibody 374B, 1000× diluted in PBS/0.1% Tween20

9. Incubate for 30 minutes at room temperature, with agitation

10. wash five times with TBS/0.1% Tween20

11. Fill wells with 3000× diluted horse raddish peroxidase-labeled polyclonal rabbit anti-mouse antibody (RAMPO, DAKOCytomation)

12. Incubate for 20 minutes at room temperature, with agitation

13. Wash five times with TBS/0.1% Tween20

14. Wash twice with PBS

15. Stain with 100 μl TMB, stop with 50 μl 10% H₂SO₄

16. Read absorbance at 450 nm

Ellagic acid was co-incubated with 0.5 nM tPA. Controls: 1 mM ThT with 0.5 nM tPA, 0.5 mM CR with 0.5 nM tPA, 0.5 nM tPA without compound and buffer without tPA. Next, coated ELISA plates were first -pre-incubated with ellagic acid (for 30 minutes) followed by tPA incubation (for 30 minutes). For similar ELISA's with DOVA and Aβ(1-40) E22Q, 1 μg/ml DOVA or Aβ was coated and overlayed with 80 nM tPA. Subsequently, DOVA or Aβ coated wells and buffer-coated control wells were first incubated with 500 μg/ml ellagic acid, followed by an overlay with tPA.

Binding of Misfolded Proteins from Solution to Immobilized Ellagic Acid

To test the ability of ellagic acid to bind to misfolded proteins, ellagic acid was immobilized at 100 μg/ml in the wells of a 96-wells Greiner Microlon high binding plate and a Nunc Maxisorp plate. Ellagic acid was coated in 100 mM NaHCO₃ pH 9.6, 50 μp/well, 1 h at room temperature with agitation. As a control for subtraction of background signals of binding of misfolded proteins to wells without ellagic acid, wells were coated with buffer only. After blocking of the plates, wells were overlayed with solutions of 0.1/1/10 μg/ml Hb-AGE or 10/100 μg/ml Aβ, in binding buffer (PBS/0.1% Tween20). After washing, binding of Hb-AGE was assessed by overlaying wells with 1 μg/ml hybridoma antibody 4B5, which binds to glycations (¹), followed by RAMPO. Binding of Aβ was visualized using 500× diluted anti-Aβ antibodies (mouse antibody beta-amyloid Clone 6F/3D #M0872, lot 00003503, DAKOCytomation; β-amyloid (H-43) SC-9129, 200 μg/ml rabbit polyclonal IgG, Santa Cruz Biotechnology) and RAMPO/SWARPO in a 1:1 ratio. Finally, wells were overlayed with OPD/H₂O₂ solution, and H₂SO₄, before absorbance readings at 490 nm.

Methods: Analysis for the Presence of Amyloid-Like Misfolded Protein Conformation at the Surface of Activated Platelets During Aggregation

The presence of protein(s) and/or peptide(s) with amyloid-like misfolded protein conformation on activated blood platelets was tested with washed platelets in an aggregometric assay. Freshly drawn human aspirin free blood was mixed gently with citrate buffer to avoid coagulation. Blood was spinned for 15 minutes at 150*g at 20° C. and supernatant was collected; platelet rich plasma (PRP). Buffer with 2.5% trisodium citrate, 1.5% citric acid and 2% glucose, pH 6.5 was added to a final volume ratio of 1:10 (buffer-PRP). After spinning down the platelets upon centrifugation for 15 minutes at 330*g at 20° C., the pellet was resuspended in HEPES-Tyrode buffer pH 6.5. Prostacyclin was added to a final concentration of 10 ng/ml, and the solution was centrifuged for 15 minutes at 330*g at 20° C., with a soft brake. The pellet was resuspended in HEPES-Tyrode buffer pH 7.2 in a way that the final platelet number was adjusted to 200,000/μl. Platelets were kept at 37° C. for at least 30 minutes, before use in the assays, to ensure that they were in the resting state.

For the aggregometric assays, 400 μl platelet solution was added to a glass tube with 100 μl containing the agonist of interest, fibrinogen and CaCl₂. Final concentrations of fibrinogen and CaCl₂ were 0.5 mg/ml and 3 mM, respectively. A stirring magnet was added and the apparatus (Whole-blood aggregometer, Chrono-log, Havertown, Pa., USA) was blanked. Aggregation was followed in time by measuring the absorbance of the solution, that will decrease in time upon platelet aggregation. As a positive control, 0.5 U/ml thrombin was used, or TRAP. Aggregation was followed for 10 minutes. Influence of 200 μM Thioflavin T, 200 μM Congo red or 1 μM tPA on platelet aggregation was analyzed. Furthermore, influence of tPA on TRAP induced platelet aggregation that was maximally inhibited with Indo (indomethacin; aspirin-like) and AR-C6993MX (clopidogrel-like), was assessed. Indo and AR-C6993MX do not fully abolish TRAP-induced platelet aggregation, so the role of amyloid-like protein conformation on this residual aggregating potential was studiesdby analyzing the influence of misfolded protein binding tPA.

Binding capacity of amyloid-specific dye Thioflavin T to activated platelets was also analyzed using standard FACS analyses. FACS analysis is performed with platelets that are stimulated with or without thrombin (1 min, 37° C.) in the presence of EDTA. The fluorescent amyloid dye Thioflavin T is used to detect amyloid on the surface of platelets.

Methods: Platelet Adhesion Experiments: Binding of Amyloid-Dye Congo Red

Blood anti-coagulated with one-tenth volume of 0.13 M sodium citrate was collected from healthy volunteers (with informed consent) who had not taken any medication during the last 10 days. Perfusion experiments were performed over glass coverslips sprayed with type III collagen (0.3 mg/ml) or coated with von Willebrand factor (vWf) (1 mg/ml), using a parallel plate perfusion chamber. Whole blood was prewarmed at 37° C. and recirculated through the perfusion chamber with a shear rate of 800/second¹ for 5 minutes. After perfusion, cover-slips were rinsed with HEPES-Tyrode buffer (pH 7.2). A Congo red Staining kit (catalogue number HT-60, Sigma, St. Louis, Mo., USA) was used according to protocol for staining the cover-slips. The cover-slips were mounted with Vectashield (Vector Labs, Burlingame, Calif., USA). Cover-slips were analyzed with a Leitz DMIRB fluorescence microscope, with a 63·Planapo objective (Leica, Voorburg, the Netherlands), interfaced with a Leica TCS4D confocal laser microscope (Leica Lasertechnik, Heidelberg, Germany).

Methods: Platelet Ageing and Amyloid-Like Misfolded Protein Conformation Formation

Platelets were isolated according to the description given above, and brought to 200.000 platelets/μl in HEPES-Tyrode buffer with 1 mM Ca²⁺. The mean platelet volume was 9.5 femtoliter. Isolated platelets were split in two portions of 5 ml. To one portion, 1 μM final concentration of A23187 (Ca²⁺Ionophore, used to mimic direct platelet ageing) was added. Both fractions were incubated at 37° C. for 10 minutes and assayed for amyloid structure by analysis of enhancement of Congo red and Thioflavin T fluorescence (see below). Furthermore, the potency of the platelets to bind and activate tPA in a chromogenic tPA/plasminogen activation assay was assessed. The conversion of chromogenic plasmin substrate PNAPEP1751 by plasmin was kinetically measured at 37° C. on a Spectramax340 microplate reader at a wavelength of 405 nm. The assay mixture contained 400 μM tPA, 100 μg/ml plasminogen (purified from human plasma) and 415 μM PNAPEP1751 in HEPES buffered saline (HBS) pH 7.4. Denatured γ-globulins (20 μg/ml) comprising amyloid-like structure was used as reference and positive control. Negative control was H₂O. The final platelet density was 50.000 platelets/μl.

Methods: Thioflavin T and Congo Red Fluorescence Measurements

Fluorescence of Thioflavin T (ThT; Sigma T-3516, St. Louis, Mo., USA) and Congo red (CR; Aldrich Chemical Company Inc., Milwaukee, Wis., USA) was measured on a Hitachi F-4500 spectrophotometer at an excitation wavelength of 435 nm and emission wavelength of 485 nm for ThT and an excitation wavelength of 550 nm and emission wavelength of 595 nm for Congo red. Twentyfive μl of both platelet suspensions were diluted in either 1 ml of 25 μM ThT in 50 mM Glycine buffer pH 9.0, or in 1 ml of 25 μM CR in phosphate buffered saline (PBS) pH 7.2, and incubated for 30 minutes at room temperature. Fluorescence was measured in triplicate. Background fluorescence of both protein in buffer and dye-solution were subtracted from the total fluorescence signal. Five μg/mL fibrillar amyloid-β(1-40) E22Q (Peptide Synthesis Facility of the Dutch Cancer Institute NKI, Amsterdam, The Netherlands) was used as a positive control in all fluorescence assays.

Results:

Protein Expression and Purification.

The proteins tPA F-EGF, Fn F4,5 and BiP were expressed to high final concentrations in the medium of HEK293E cells. Subsequent purification using Ni-Sepharose resin resulted in 80-90% purity, as observed on SDS-PAGE gel. Resulting protein samples were dialysed and tested for their affinity for several misfolded proteins (described below). The proteins were coupled to Ni-Sepharose beads to prepare affinity matrices that were used for misfolded protein depletion (“Fish”) experiments.

Binding Affinities of BiP, Fibronectin F4,5 and tPA F-EGF for Misfolded Proteins

In a first test, binding of tPA F-EGF, BiP and Fn F4,5 to glycated BSA was analysed (FIG. 7A). Next, BiP was tested for its affinity for BSA-AGE or heat-denatured BSA versus BSA, Hb-AGE versus Hb (FIG. 7B, C). It was found to bind to BSA-AGE with a high affinity, but not to freshly dissolved BSA or heat-denatured BSA. It also bound Hb-AGE, but not freshly dissolved Hb.

The affinity of fibronectin F4,5 for several misfolded proteins and their native controls was tested in an ELISA setup (FIG. 7D-H). High affinities for AGEs (BSA-AGE and Hb-AGE) were found, whereas the affinity for their native controls was very low. A clear difference in binding affinity for heat-denatured OVA versus freshly dissolved OVA was observed, whereas reduced and alkylated OVA acted as its native control. Amyloid γ-globulins (denatured at 37° C.) was able to bind Fn F4,5 with high affinity, whereas freshly dissolved γ-globulins and alkyl-γ-globulins had low affinity for Fn F4,5. Freshly dissolved lysozyme as well as reduced and alkylated lysozyme both showed high affinity for Fn F4,5.

Finally, tPA-F EGF, that was purified using Ni-Sepharose, binding to misfolded protein was tested. In the subsequent dialysis step, most protein precipitated. The remaining soluble protein was tested for its affinity to several misfolded proteins. High binding affinities for BSA-AGE relative to its native control was observed (FIG. 7A). Much lower binding to heat-denatured OVA relative to freshly dissolved OVA was observed (FIG. 7I).

Misfolded Protein Extraction Experiments

The purified and dialysed proteins BiP, fibronectin F4,5, tPA F-EGF and sRAGE, all with a carboxy-terminal His-tag were bound to Ni-Sepharose to obtain an affinity matrix for binding of misfolded protein. Samples with or without a 0.5 μg/ml spike of BSA-AGE were incubated with the affinity matrices. Depletion of the solutions from BSA-AGE by the affinity matrix was analysed in an ELISA (FIG. 8). BSA-AGE was extracted from three solutions: PBS, 256-fold diluted serum in PBS and 512-fold diluted plasma in PBS, all in the presence of 0.1% Tween20 and 20 mM imidazole.

When comparing residual BSA-AGE content in a solution that was incubated with empty control Ni-Sepharose beads, with BSA-AGE starting solution, the control beads did not bind BSA-AGE (FIG. 8). Incubation of BSA-AGE in PBS, diluted serum or diluted plasma with either of the four affinity matrices revealed that fibronectin F4,5 and sRAGE (FIG. 8) were more efficient misfolded protein binding moieties for depletion of the solutions from BSA-AGEI than BiP and tPA F-EGF. Both Fn F4,5-Ni Sepharose and sRAGE-Ni Sepharose beads extracted the 0.5 μg/ml BSA-AGE almost completely from the solution.

These results show that proteins and protein domains that are natural misfolded protein binding moieties are suitable for being implemented in misfolded protein depletion/isolation technology. Further refinement of the choice of immobilization of the binding moieties and the incubation parameters will direct the technology towards even more efficiency and specificity. Based on the requirements, the misfolded protein binding moieties are immobilized on a suitable solid support of choice. Based on the application, binding conditions are adjusted. Based on the misfolded protein ligand that has to be depleted, the misfolded protein binding moiety is chosen and refined. For example, when depletion of a biopharmaceutical from misfolded constituents including misfolded biopharmaceutical itself, is required, binding conditions are driven by the excipients combination of the biopharmaceutical. Adjustable parameters are still the type or combination of types of misfolded protein binding moieties, the incubation time, the incubation technique (batch wise, (linear/circulating) flow), temperature, type of support with the binding moiety etcetera.

I. Coupling of tPA F-Biotin to Streptavidin-Sepharose

To analyze whether tPA F-biotin is coupled to Streptavidin-Sepharose beads, solution after coupling and wash buffer was analyzed for the presence of tPA F-biotin in a direct ELISA with coated dilution series of solutions with tPA F-biotin and a tPA F-biotin standard. A representative curve for the dilution series of tPA F-biotin before and after contacting Streptavidin-Sepharose is shown in FIG. 9A. The ELISA analysis of the tPA F-biotin coupling efficiency revealed that approximately 44% of the tPA F-biotin is coupled to Streptavidin-Sepharose. This has resulted in a tPA F-biotin density of approximately 1.5 μg/μl beads. Coupling was also verified by analyzing beads on Western blot (not shown). When comparing with a standard tPA F-biotin dilution series, it is concluded that indeed approximately 0.25-1.25 μg F-biotin is coupled per μl beads.

II. Depletion of Buffer or Plasma from Misfolded Protein upon Contacting with tPA F-Biotin—Streptavidin-Sepharose

Similarly to the experiments with BiP—Ni-Sepharose, tPA F-EGF—Ni-Sepharose, fibronectin F4,5—Ni-Sepharose and sRAGE—Ni-Sepharose, diluted plasma and buffer was spiked with 1 μg/ml BSA-AGE and contacted to tPA F-biotin—Streptavidin-Sepharose, and the supernatant was subsequently analyzed for the remaining fraction of BSA-AGE. The control was unspiked buffer or plasma, and Streptavidin-Sepharose without misfolded protein affinity ligand. FIG. 9B shows the results of a sandwich ELISA for detection of BSA-AGE in solution. It can be clearly seen that upon contacting buffer or diluted plasma with BSA-AGE spike, most of the BSA-AGE is specifically extracted from the solutions, when compared to starting solutions. Control beads do not exert any effect on the amount of BSA-AGE in solution.

In a next experiment, 512-fold diluted human single donor plasma was spiked with 250 μg/ml BSA-AGE and platelet activating properties of a tenfold diluted solution was analyzed (FIG. 9C). Platelets readily aggregate upon contacting the misfolded protein. The diluted plasma with BSA-AGE spike was also contacted to tPA F-biotin—Streptavidin-Sepharose, which is an affinity matrix for misfolded proteins. After incubation for 2 hours, supernatant was analyzed for platelet activating potential. As seen in FIG. 9C most of the platelet activating potential has been efficiently removed by the tPA F-biotin—Streptavidin-Sepharose. By removal of BSA-AGE from plasma, the pro-thrombotic activity of the solution comprising the amyloid-like misfolded protein is strongly reduced. This shows that removal of misfolded protein from solution is beneficial with respect todverse effects on cells. With the current parameters used, it is now possible to refine the depletion technology towards the required conditions for a specific application. Furthermore, depletion of plasma from misfolded proteins can be optimized by adjusting parameters like for instance incubation buffer, time, temperature, affinity ligand, solid support/type of matrix, and more.

RESULTS: Binding of LRP Cluster IV to Misfolded Protein

Human extracellular LRP fragment cluster IV was successfully cloned from THP-1 cell DNA, and subsequently expressed in HEK 293E cells. On a Western blot, protein with the expected molecular weight was detected upon incubation of the nitrocellulose blot membrane with anti-FLAG-tag antibody (not shown).

To analyze the property of the expressed LRP cluster—IV-FLAG protein to bind to misfolded protein, binding was assessed using an ELISA set-up with coated misfolded glycated albumin and haemoglobin, and their freshly dissolved lyophilized non-glycated counterparts. As can be seen in FIG. 10A, LRP cl-IV binds specifically to BSA-AGE as well as to Hb-AGE, and not to the freshly dissolved BSA and Hb.

Now that specific binding of LRP cluster IV to amyloid-like BSA-AGE was established, we wondered whether known amyloid-binding moieties tPA, ThT, ThS and Congo red influence the binding. This would further show the involvement of the amyloid-like misfolded protein conformation in binding of LRP or in inducing the LRP binding site. As can be seen in FIG. 10B, C, tPA, K2P tPA and ThT at the assay conditions and concentrations tested do not interfere with binding of LRP cl-IV to BSA-AGE. Congo red and ThS, however do inhibit binding of LRP cl-IV to BSA-AGE to a large extent (FIG. 10D, E). This shows that amyloid-binding dyes Congo red and ThS bind to, or close to the binding site of LRP for misfolded proteins. Apparently, tPA and ThT may bind to a different feature of the misfolded BSA-AGE. This makes LRP to a valuable tool for incorporation in development programs of technology for depletion of misfolded protein from solution. Depending on the application and the targeted misfolded protein(s), LRP is a preferred misfolded protein binding moiety, next to, as alternative for, or in combination with other identified moieties with affinity for amyloid-like misfolded proteins.

Results of Structural Analyses of Misfolded β2gpi

Exposure of human native β2gpi to cardiolipin, or alkylation of cysteine residues in β2gpi induces amyloid-like protein conformation (FIG. 11). This is detected with the known amyloid-specific dye Thioflavin T, as well as with the natural misfolded protein binding serine protease tPA.

Results

Amyloid-Like Protein Conformation in Protein Solutions Applied as Biopharmaceuticals

Over the past decades, the use of therapeutic proteins has become common practice in medicine and as their use is very promising, many more biopharmaceuticals are under development. Unfortunately, a major drawback of protein therapeutics is the risk of antibody formation. These immunogenicity problems are of concern regarding therapeutic efficacy and patient safety. Protein misfolding is an intrinsic and problematic property of proteins, which underlies a variety of degenerative diseases, such as Alzheimer's disease. These diseases are characterized by the occurrence of fibrillar deposits, termed amyloid, containing aggregates of misfolded proteins. The common denominator in amyloids is the cross-β structure. While the term amyloid is used to classify these fibrillar deposits, aggregation of proteins, irrespective of amino-acid sequence, results in formation of amyloid-like properties with similar features. Protein misfolding can be accelerated by a number of environmental factors, including protein modifications such as glycation, deamidation or oxidation, interaction of proteins with surfaces, such as mica or negatively charged phospholipids or other conditions, such as heating, lyophilization, sonication, packaging materials.

We now show that misfolding of therapeutic proteins also leads to the formation of amyloid-like properties and that this underlies the triggering of antibody formation. These data serve as prototype examples for the identification of the presence of misfolded protein molecules in protein solutions, preferably therapeutic protein solutions.

We examined whether proteins with amyloid-like properties are present in marketed biopharmaceuticals. As indicators for amyloid-like properties we measured the fluorescence of Thioflavin T (ThT), Congo Red and binding and activation of tissue-type plasminogen activator (tPA), all qualitative measures for the presence of amyloid-like misfolded protein conformation in proteins in solution. As shown in Table 4, several biopharmaceuticals showed significant potential to enhance fluorescence of Thioflavin T and/or Congo Red, indicating the presence of amyloid-like structure. These biopharmaceuticals also bound to bind tPA with high affinity and activated tPA-mediated plasminogen activation (Table 4). These findings demonstrate that amyloid-like properties are present in various marketed therapeutic proteins.

Most protein pharmaceuticals can be stored for prolonged periods of time without losing their bioactivity. However, some fraction of proteins gradually looses its structure and degrades. We examined the effect of storage on the level protein with amyloid-like structure in a number of biopharmaceuticals. FIG. 12 shows that the level of protein with amyloid-like properties increases when the biopharmaceuticals were examined closer to their expiration date.

During manufacturing and storage, biopharmaceuticals also become exposed to various conditions of stress that potentially underlie the formation of amyloid-like properties. To artificially mimic stability testing we examined whether exposure of biopharmaceuticals to conditions of severe stress, such as low pH are heat, induced amyloid-like properties. FIG. 13 shows that amyloid-like properties are adopted by Etanercept, Glucagon, Abciximab, and Infliximab upon exposure to these harsh denaturing conditions. Thus, like any protein, biopharmaceuticals adopts similar amyloid-like properties and this is enhanced upon storage or under conditions of stress.

Pharmaceutical compositions with amyloid-like properties are responsible for enhanced immunogenicity of biopharmaceuticals and breaking of tolerance. Here we disclose a unifying mechanism by which individual immunogenic factors, such as oxidation or formulation changes, result in adoption of amyloid-like properties, ultimately leading to immune responses.

In summary, our technology provides a method for detecting a protein and/or peptide comprising an amyloid-like misfolded protein conformation in an aqueous solution comprising a protein, said method comprising: contacting said aqueous solution comprising a protein with at least one cross-β structure-binding compound resulting in a bound protein and/or peptide comprising a cross-β structure, and detecting whether bound proteins and/or peptides comprising a cross-β structure are present in said aqueous solution. Said aqueous solution comprising a protein, for instance comprises a detergent, or a food and/or a food supplement, or consists of a cell culture medium, or a commercially available protein, or protein/peptide solution used for research purposes, or blood and/or blood product, or a cosmetic product, or a cell, or a combination of any of these. Furthermore, we provide examples of a method for controlling a manufacturing process, and/or storage process of an aqueous solution comprising a protein, said method comprising: contacting said aqueous solution with at least one cross-β structure-binding compound resulting in a bound protein or peptide comprising a cross-β structure, detecting whether bound proteins and/or peptides comprising a cross-β structure are present in said aqueous solution at various stages of said manufacturing and/or storage process.

Use of Ellagic Acid in Technology for Depletion of Protein Solutions from Misfolded Proteins.

Results

Binding of tPA to Misfolded Proteins; Influence of Ellagic Acid

To show that ellagic acid is a compound with the ability to interact with amyloid-like misfolded protein, the influence of ellagic acid was analyzed twice on the binding of tPA to immobilized Hb-AGE. Ellagic acid was dissolved at 5 mg/ml in DMSO. To be sure that the interaction of tPA with the misfolded protein is not driven by the tPA Kringle domains, 10 mM εACA, a compound that abolishes interaction of Kringle domains with lysine and arginine residues, was always included in the binding buffer. The tPA and ellagic acid were mixed before being pplied to an ELISA plate well. The binding of tPA from solution without ellagic acid to immobilized Hb-AGE was set to 100%. See FIG. 14A for binding data in duplicate. With the used method, it was not yet established whether ellagic acid interacts with tPA or with immobilized misfolded protein. Therefore, ellagic acid was first exposed to immobilized Hb-AGE, followed by a tPA incubation. In this way, direct interaction with the immobilized misfolded Hb-AGE is shown. In a similar way, ellagic acid was first added to wells with coated misfolded OVA, followed by tPA overlays (FIG. 14B). These combined binding studies revealed that ellagic acid binds directly to misfolded protein. In a next series of more detailed experiments using triplicate overlays of wells instead of single-well overlays, and using buffer-coated wells for background compound/tPA signal subtraction, concentration series of ellagic acid were applied to immobilized Aβ, Hb-AGE or misfolded OVA, followed by an overlay with a sub-optimal concentration of tPA (FIG. 14C-E).

Binding of Misfolded Hb-AGE and Aβ from Solution to Immobilized Ellagic Acid

To test the ability of ellagic acid, that influences binding of tPA to immobilized misfolded proteins, to extract misfolded protein from solution when ellagic acid was fixed to the wells of an ELISA plate, ellagic acid was coated to Greiner Microlon high-binding 96-wells plates, Nunc Maxisorp plates and Nunc amino Immobilizer plates, and overlayed with concentration series of amyloid-β or glycated haemoglobin. Hb-AGE binding was observed with ellagic acid on a Nunc Maxisorp ELISA plate and on a Greiner Microlon high-binding plate, as has been observed consistently in duplicate experiments (Table 5). Aβ binding was observed with immobilized ellagic acid on a Nunc Maxisorp plate. We have established that ellagic acid is a stimulator of tPA binding to Hb-AGE and DOVA. Therefore, it is concluded that ellagic acid interacts with misfolded proteins that are immobilized in the wells of an ELISA plate, as well as vice versa with the misfolded protein in solution/suspension and ellagic acid immobilized on ELISA plates. The ability of ellagic acid to extract misfolded protein from solution makes it a lead candidate for development of affinity matrices for misfolded proteins, that are suitable for being applied for purification methods aimed at depletion of solutions from harmful misfolded proteins.

When immobilized on a suitable carrier, ellagic acid is able to bind misfolded protein from solution. This result provides a preferred example of a method for at least partly removing from a solution an amyloid-like misfolded protein comprising contacting said solution with a compound capable of binding to misfolded protein and/or with a compound capable of binding to a protein conformation induced by misfolding in a protein, and removing the resulting complex from said solution.

Results: Identification of Amyloid-Like Misfolded Protein at the Surface of Activated Human Blood Platelets

Blood Platelets Express Amyloid-Like tPA-Philic Structures Upon Activation and Show Increased Binding of Amyloid Dye Thioflavin T and Misfolded Protein Binding tPA Upon Aging

Recent insights have indicated that the formation of amyloid is not necessarily the result of a defect in the normal folding or clearance pathway, but that amyloid is also formed through normal biological proteolytic processing. We found that (i) activation of platelets induce amyloid at their cell surface and (ii) that platelets adhered to von Willebrand factor (vWF) or collagen surface under flow express amyloid domains (FIG. 15A-D). Expression is at the cell body and areas of spreading are negative (vWF surface) and at tips of aggregates (collagen) indicating that adhered and aggregated platelets express areas rich and poor in amyloid. Platelets stimulated with TRAP (an activator of the PAR-1 receptor without proteolytic properties and incapable ofconverting released fibrinogen into fibrin) and thrombin (an activator of PAR-1 and PAR-4 through proteolysis and an activator of fibrin formation) express amyloid as visualized using the fluorescent amyloid dyes Congo red (not shown) and ThT in a FACS analysis (FIG. 15C, D). Resting platelets do not express amyloid at the cell surface (FIG. 15C).

To test whether amyloid influences platelet aggregation by classical stimuli, we tested whether amyloid specific dyes and the amyloid binding protein tPA affect platelet aggregation (FIG. 15E, F). Indeed, using optical aggregometry we observed that Congo red, ThT as well as tPA inhibited platelet aggregation. Dose response studies show up to 30% inhibition by 200 μM Congo red and up to 45% inhibition by 200 μM ThT (FIG. 15E). tPA (1 μM) even induced 55% inhibition of thrombin-induced aggregation. The inhibition persisted in platelets treated with indomethacin (aspirin-like) and AR-C6993MX (clopidogrel-like), indicating that amyloid contributed to platelet aggregation via mechanisms independent of thromboxane A₂ formation or P2Y12 stimulation through released ADP (FIG. 15F).

Further proof for the presence of proteins with amyloid-like protein conformation was revealed by analyzing platelets in time for the presence of misfolded protein, using binding of amyloid-binding moieties as a read-out (FIG. 15G-I). Platelets display increased ThT binding upon ageing (FIG. 15G). In addition, rapidly induced ageing/activation by adding ionophore A23187 to the platelet suspension resulted in increased ThT, Congo red and tPA binding (FIG. 15G-I). Subsequent ageing after addition of the ionophore did hardly induce further appearance of misfolded protein at the surface of the platelets. These combined results show that our techniques for identification of the presence of amyloid-like misfolded proteins on cells provide evidence for the induction of amyloid-like protein misfolding upon ageing and/or activation of platelets. This is for instance helpful for stability testing of stored platelets meant for transfusion purposes. Analysis of the correlation between the amount of misfolded protein detected with the depicted assays, and the risks for adverse effects in patients receiving the platelets will provide additional understanding of platelet biology and improves platelet transfusion risk assessment and storage technology.

In summary, we provide a method for detecting a cell comprising a protein and/or peptide with amyloid-like misfolded protein conformation on its surface in a collection of cells, said method comprising contacting said cell with a cross-B structure-binding molecule, and measuring binding of said molecule to said cell. Furthermore, our results of the amyloid depletion experiments with ellagic acid, BiP, tPA F-EGF, sRAGE and Fn F4,5 provide a method for removing a cell comprising a protein and/or peptide with amyloid-like misfolded protein conformation, optionally comprising cross-β structure on its surface from a collection of cells, said method comprising contacting said cell with an amyloid-like misfolded protein binding molecule, and binding said molecule to a solid surface.

TABLE 4
The presence of protein with amyloid-like properties in various
biopharmaceuticals.

| | Fluorescence (a.u. +/− SD) | | | | tPA activation |
|---|---|---|---|---|---|
| Therapeutic | | | Bmax | tPA Binding | Max. Activation |
| protein | ThT | CR | (OD_{450 nm}) | Kd (nM) | (%) |
| Albumin* | 1970 +/− 5 | 978 +/− 2 | 1.228 | 11.22 | 47.67 |
| Somatropin | 1317 +/− 10 | 429 +/− 2 | 0.9369 | 9.048 | 113.95 |
| Insulin Zn | 387 +/− 72 | 79 +/− 6 | 0.7558 | 105.4 | 17.44 |
| Suspension | | | | | |
| Insulin | 172 +/− 3 | 81 +/− 2 | 3.617 | 694.7 | 70.93 |
| Aspart | | | | | |
| Factor VIII* | 306 +/− 12 | 290 +/− 6 | 0.5398 | 229.8 | 4.22 |
| Abciximab | 8 +/− 8 | 25 +/− 1 | 0.5329 | 216.3 | 0 |
| Epoietin Alfa | 14 +/− 2 | 19 +/− 3 | ND | ND | 0 |
| Etanercept | 23 +/− 3 | ND | ND | ND | 0 |
| Infliximab | 19 +/− 1 | 67 +/− 2 | ND | ND | 0 |
| γ-Globulins* | 25 +/− 2 | 0 +/− 1 | ND | ND | ND |
| Glucagon | 48 +/− 1 | ND | ND | ND | 11.25 |

Content of protein with amyloid-like properties in biopharmaceuticals was determined by enhancement of Thioflavin T (ThT) and Congo red (CR) fluorescence, binding of tissue-type plasminogen activator (tPA) and tPA-dependent plasminogen activation (% of standardized positive control). Biopharmaceuticals containing the highest levels of cross-β structure are listed at the top.
(*plasma purified drug products)

TABLE 5
Extraction with ellagic acid of misfolded protein ligands
glycated haemoglobin and amyloid-β from solution

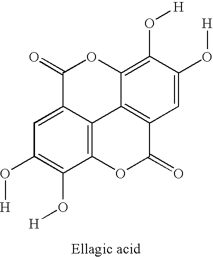

| ELISA plate | Misfolded protein | Ligand concentration | Signal |
|---|---|---|---|
| type | ligand | (μg/ml) | (a.u.)^{‡} |
| Nunc Maxisorp | Hb-AGE | 10 | 0.15 |
| Nunc Maxisorp | Aβ | 100 | 0.054 |
| Greiner | Hb-AGE | 10 | 0.13 |
| Microlon | | | |

^{‡}Background signals of 0 μg/ml misfolded protein ligand are subtracted.

### LEGENDS TO THE FIGURES

FIG. 1. Binding of polypeptides with cross-β structures to tPA, sRAGE and fibronectin type I domains, studied with Biacore surface plasmon resonance.

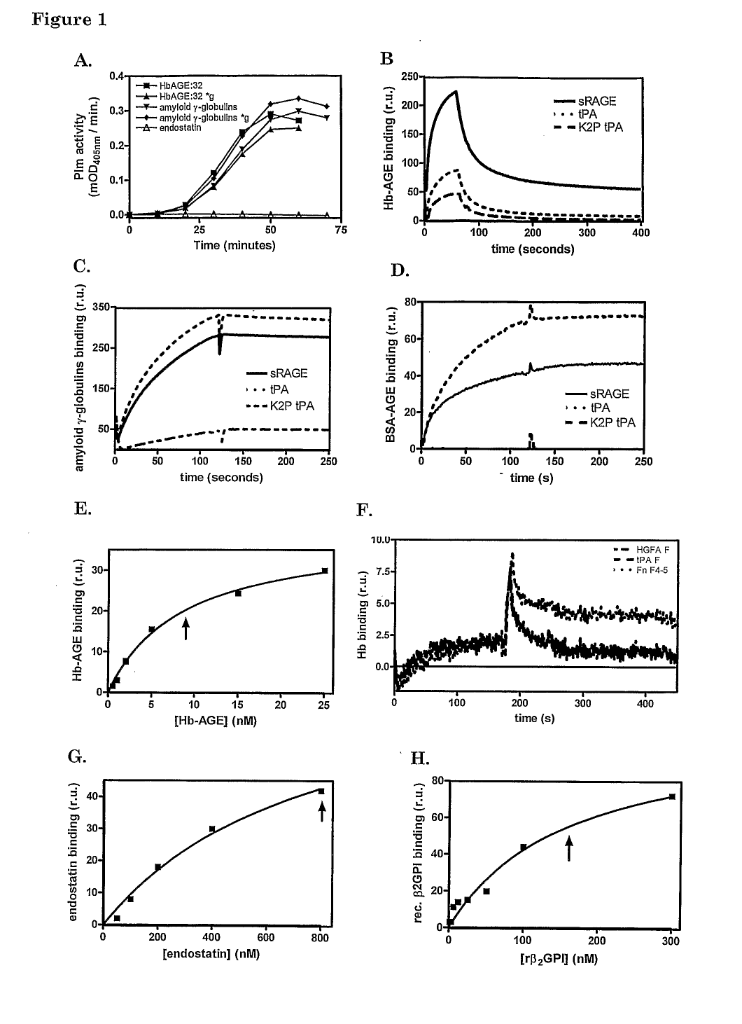

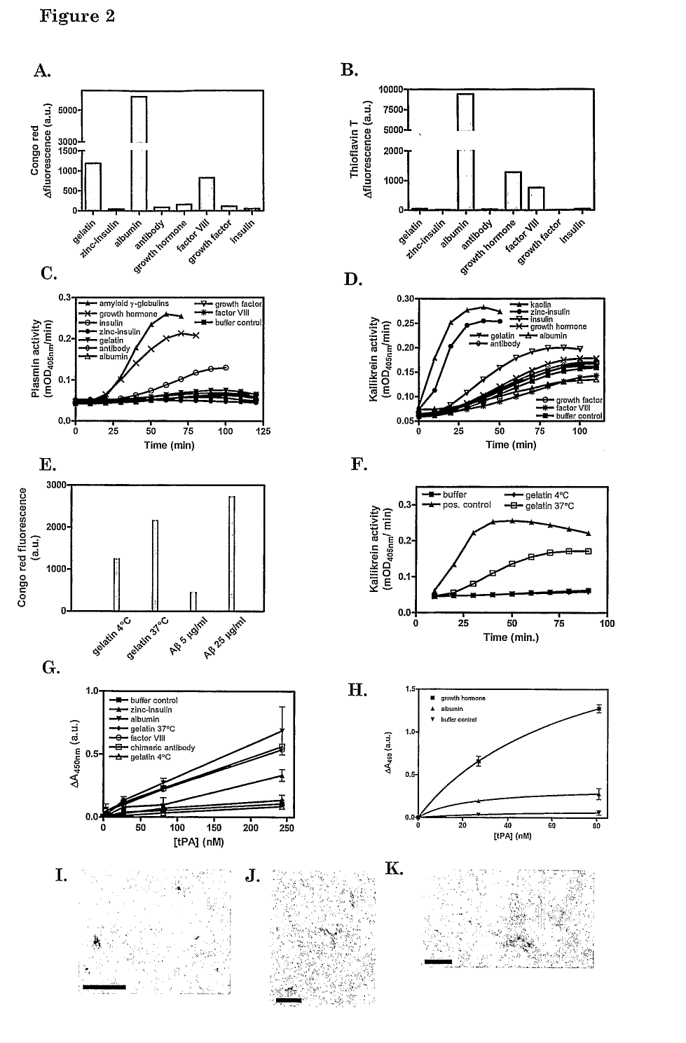

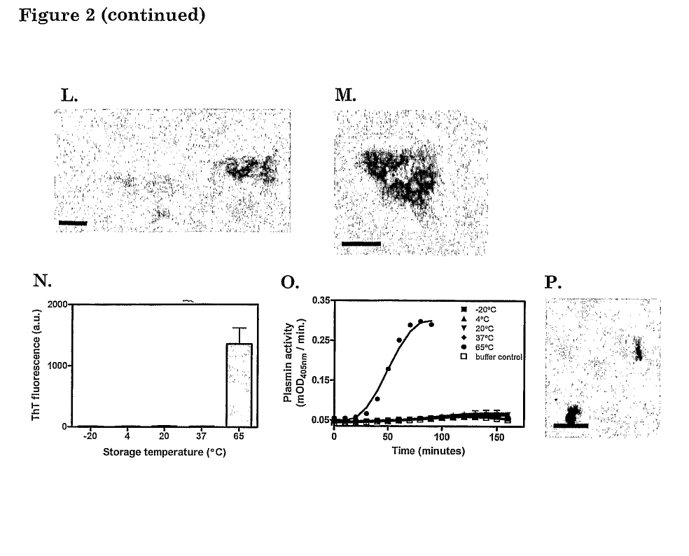

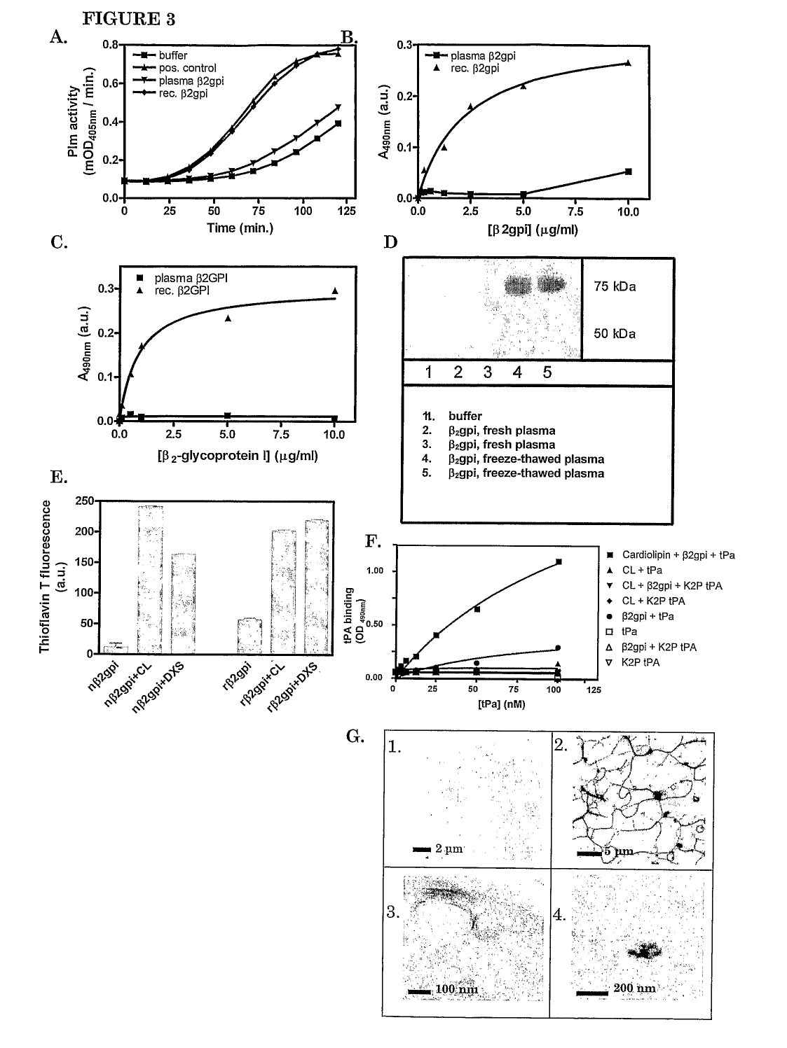

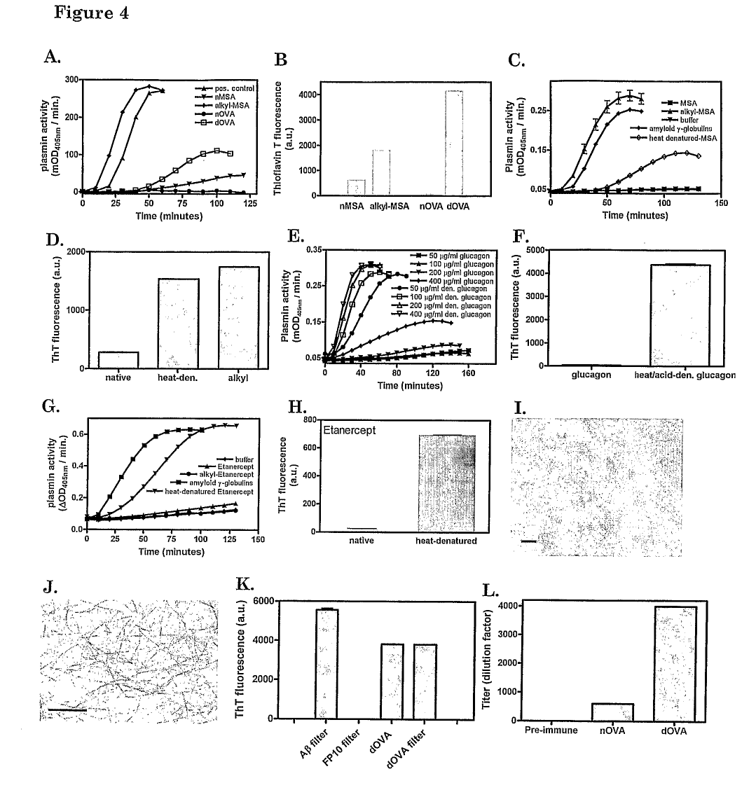

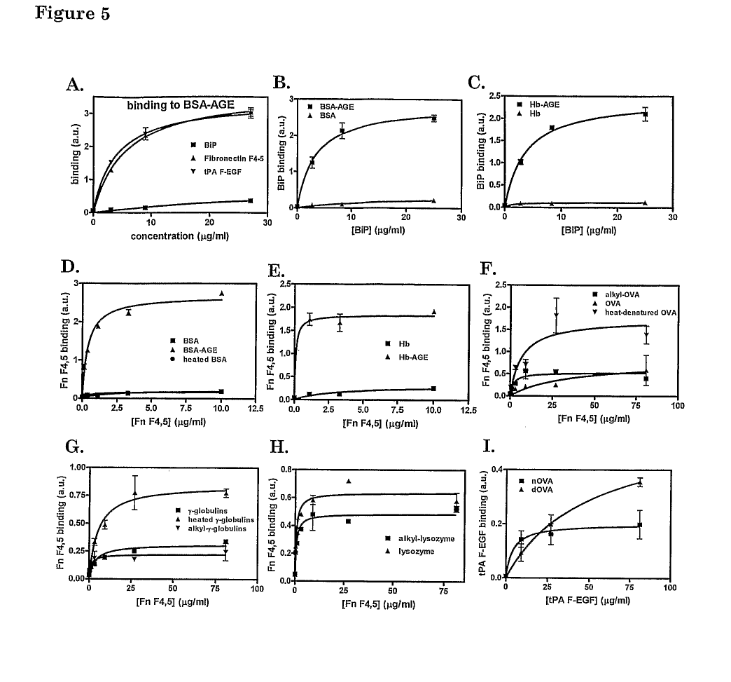

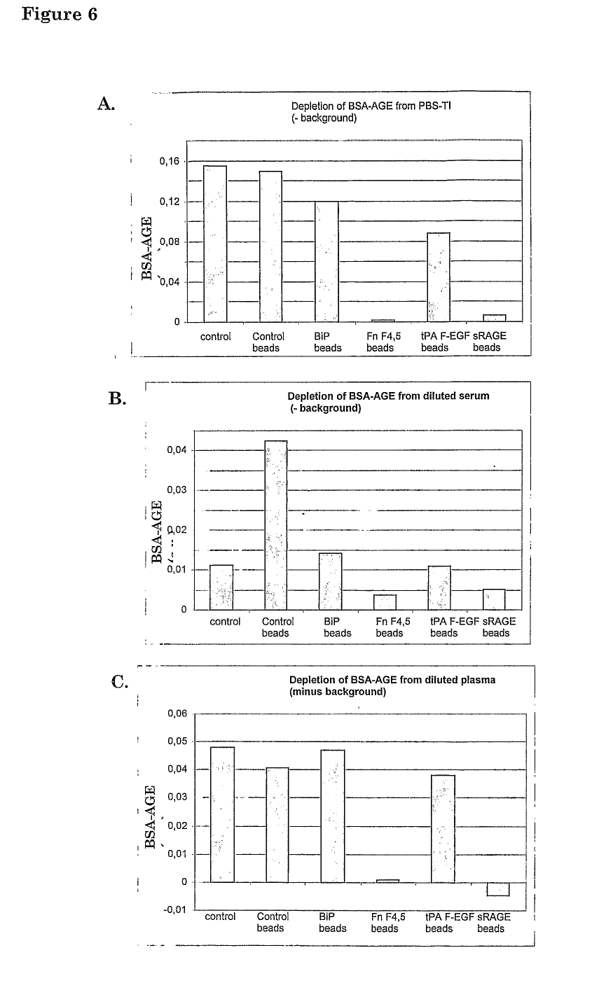

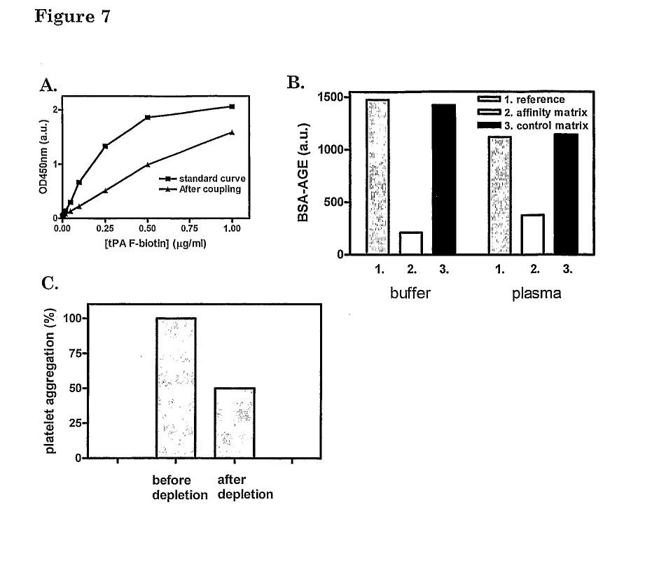

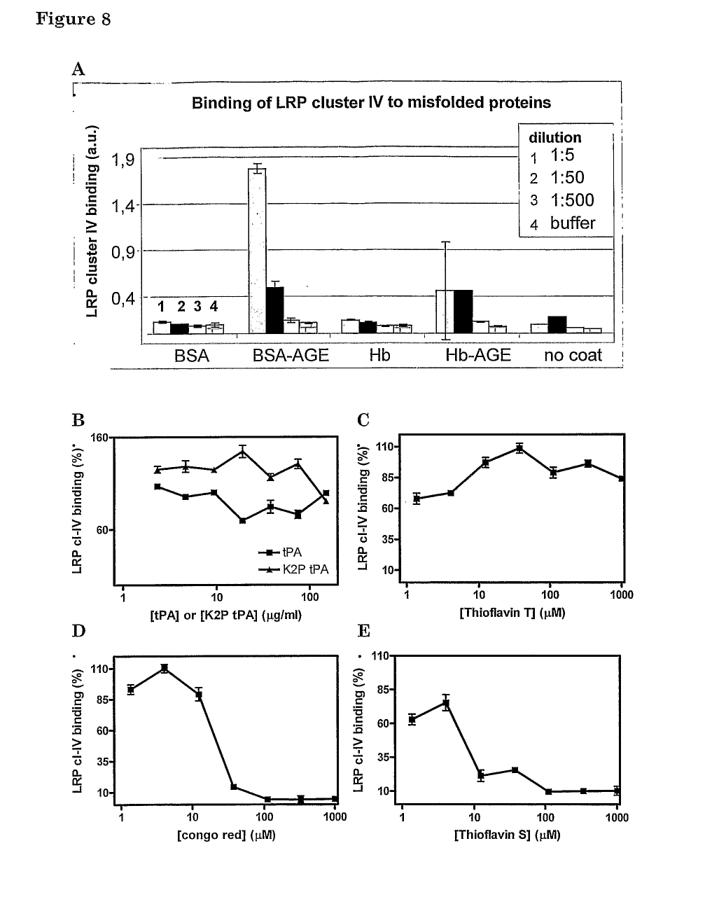

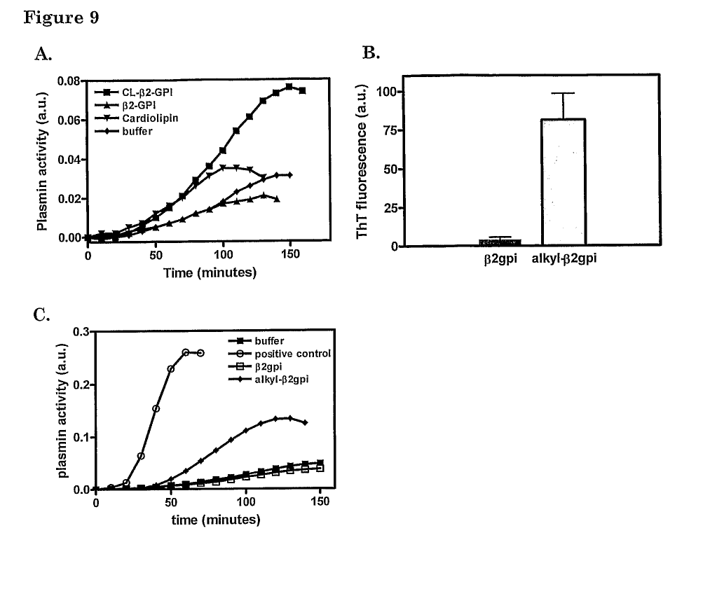

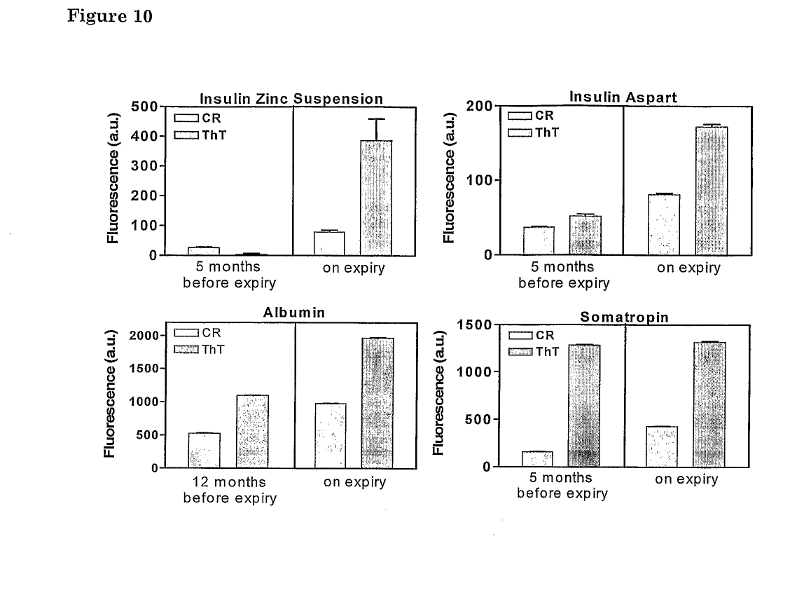

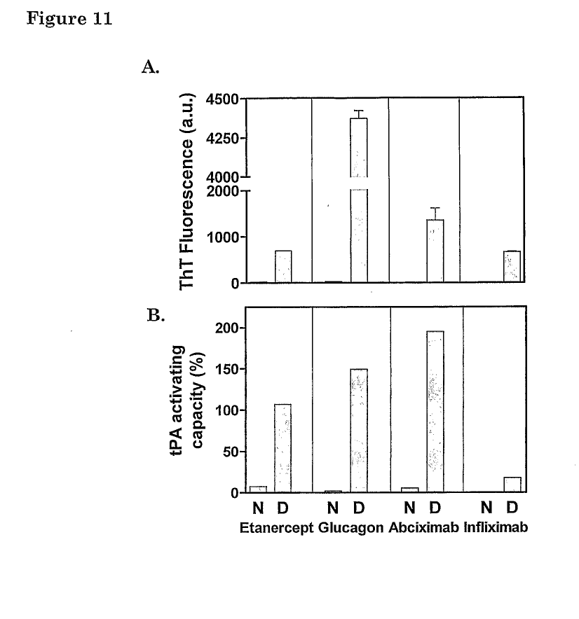

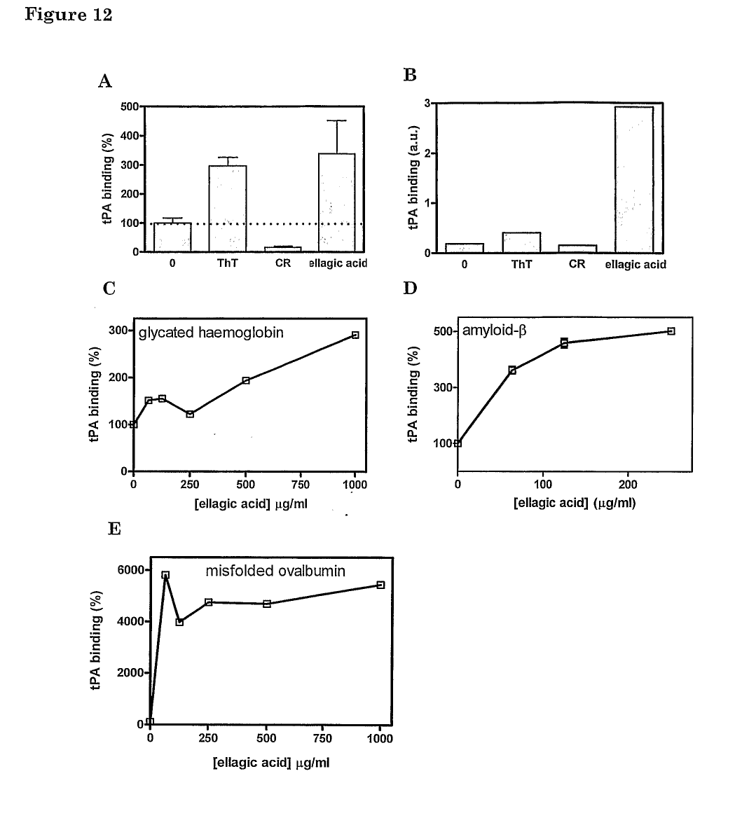

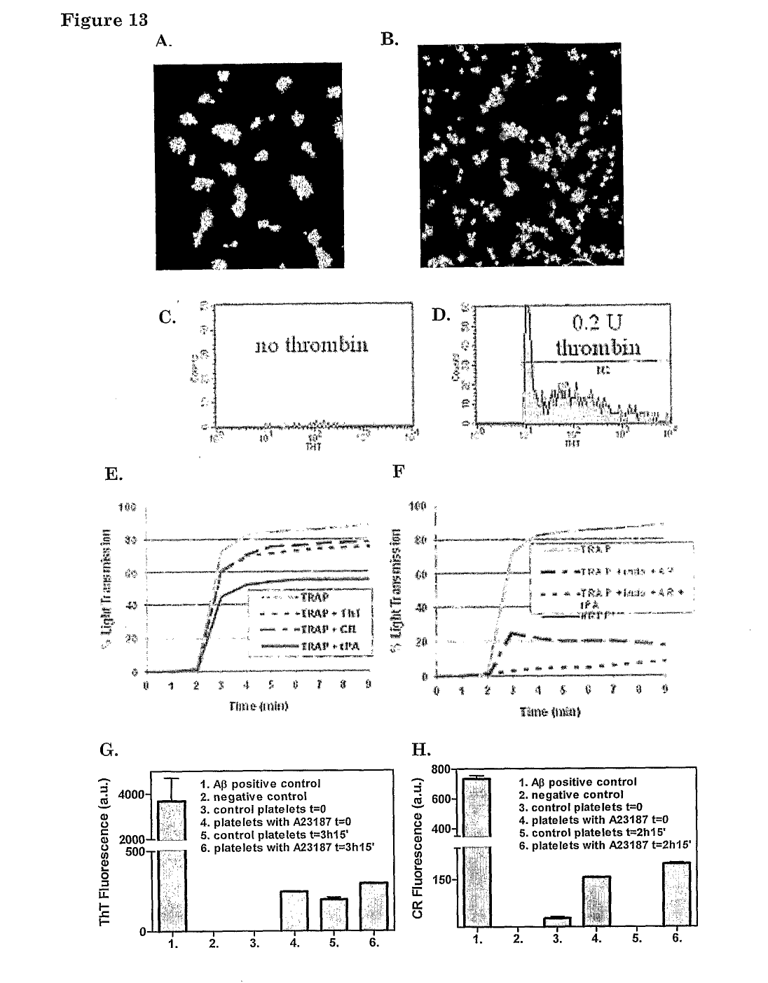

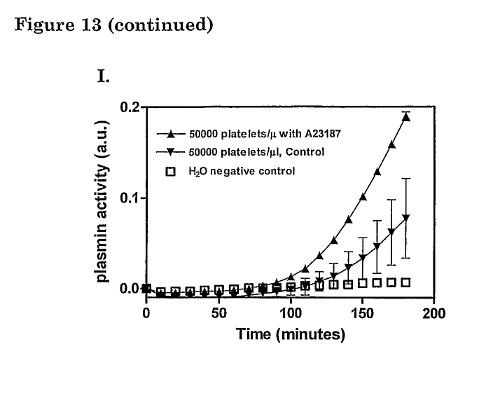

A. tPA activation assay showing that 10′ centrifugation at 16,000*g of Hb-AGE and amyloid γ-globulins hardly influences the tPA activating properties of the supernatant when compared to uncentrifuged amyloid stocks. Also protein therapeutic endostatin is tested for tPA activating properties. Concentrations of potential activators were 100 μg ml⁻¹. B. Binding of 32 μg ml⁻¹ Hb-AGE to tPA and sRAGE in a Biacore surface plasmon resnonance experiment. C. On the same chip relatively strong binding of 62.5 μg ml⁻¹ to tPA and sRAGE is observed. D. More BSA-AGE, injected at 3.9 μg ml⁻¹, binds to tPA than to sRAGE. E. By testing a concentration series of Hb-AGE for binding to a Biacore CM5 chip with immobilized Fn F4-5, it is deduced that half maximum binding is obtained with 8 nM Hb-AGE (indicated with the arrow). F. As a control, 25 nM native Hb was tested for binding to a Biacore chip with immobilized Fn F4-5, HGFA F and tPA F. G. By testing a concentration series of endostatin it is revealed that half maximum binding to Fn F4-5 is obtained with 800 nM endostatin (arrow). H. Half maximum binding of recombinant β2GPI to immobilized Fn F4-5 is obtained with 165 nM β2GPI (arrow).

FIG. 2. Presence of amyloid cross-β structures in protein solutions.

A-D. With protein solutions stored at the recommended temperature of 4° C., influence on Congo red—(A.) and ThT fluorescence (B.) was established as well as the ability to activate tPA (C.) and factor XII (D.), as determined with chromogenic assays which record Pls and kallikrein activity, that is established upon activation of Plg by tPA and prekallikrein by factor XII, respectively. Gelatin, CeaIb and FVIII clearly enhance Congo red fluorescence. CeaIb, GH and FVIII enhance ThT fluorescence. GH and insulin potentiate Plm activity. Amyloid γ-globulins at 100 μg ml⁻¹ was used as a positive control. Zinc-insulin and insulin activate factor XII. Kaolin at 150 μg ml⁻¹ was used as a positive control. E. Both modified gelatin stored at 4° C. and at 37° C. show enhanced Congo red fluorescence comparable to the positive control, 25 μg ml⁻¹ Aβ. F. Only modified gelatin that was stored at 37° C., and not gelatin stored at 4° C., exhibits factor XII stimulatory activity, as measured in a chromogenic kallikrein activity assay. The positive control for factor XII mediated prekallikrein activation was 150 μg ml⁻¹ kaolin. G. tPA ELISA showing the binding of tPA to immobilized zinc-insulin, an antibody, FVIII and albumin. Positive control in the ELISA was Hb-AGE, that is not shown for clarity. H. tPA ELISA showing the binding of tPA to immobilized Cealb and GH. K_{D}'s are 23 nM for CeaIb and 72 nM for GH. I. TEM image of modified gelatin showing various relatively condense aggregates. The scalebar is 1 μm. J. TEM image of GH showing a linear, a branched and a condense particle all apparently composed of spherical particles. The scale bar is 100 nm. K. TEM image of zinc-insulin showing the appearance of insulin as thin unbranched fibrils with varying length. The scale bar represents 100 nm. L. TEM image of CeaIb stored at 4° C. Scale bar: 100 nm M. TEM image of insulin, stored at 4° C. Scale bare: 100 nm. N. Influence of storage temperature on ThT fluorescence enhancement by Reopro. O. tPA activating properties are largely dependent on the storage temperature of Reopro, as assessed in a tPA activation assay. P. TEM image of ReoPro, stored at 4° C. Scale bar: 1 μm.

FIG. 3: Binding of factor XII and tPA to β₂-glycoprotein I.

A. Chromogenic Plg-activation assay showing the stimulatory activity of recombinant β₂GPI on the tPA-mediated conversion of Plg to Pls. The positive control was amyloid fibrin peptide FP13. B. In an ELISA, recombinant β₂GPI binds to immobilized tPA, whereas β₂GPI purified from plasma does not bind. The k_{D} is 2.3 μg ml⁻¹ (51 nM). C. In an ELISA, factor XII binds to purified recombinant human β₂GPI, and not to β₂GPI that is purified from human plasma, when purified factor XII is immobilized onto ELISA plate wells. Recombinant β₂GPI binds with a k_{D} of 0.9 μg ml⁻¹ (20 nM) to immobilized factor XII. D. Western blot incubated with anti-human factor XII antibody. The β₂GPI was purified either from fresh human plasma or from plasma that was frozen at −20° C. and subsequently thawed before purification on a β₂GPI affinity column. Eluted fractions are analyzed on Western blot after SDS-PA electrophoresis. When comparing lanes 2-3 with 4-5, it is shown that freezing-thawing of plasma results in co-purification of factor XII together with the β₂GPI. The molecular mass of factor XII is 80 kDa. E. Exposure of 25 μg ml⁻¹ β₂GPI, recombinantly produced (rβ₂GPI) or purified from plasma (nβ₂GPI), to 100 μM CL vesicles or to 250 μg ml⁻¹ dextran sulphate 500,000 Da (DXS) induces an increased fluorescence of ThT, suggestive for an increase in the amount of cross-β structure in solution. Signals are corrected for background fluorescence of CL, DXS, ThT and buffer. F. Binding of tPA and K2P tPA to β₂GPI immobilized on the wells of an ELISA plate, or to β₂GPI bound to immobilized CL is assessed. β₂GPI contacted to CL binds tPA to a higher extent than β₂GPI contacted to the ELISA plate directly. K2P tPA does not bind to β₂GPI. TPA does not bind to immobilized CL. G. Transmission electron microscopy images of 400 μg ml⁻¹ purified plasma β2GPI alone (1) or contacted with 100 μM CL (2, 3) and of 400 μg ml⁻¹ purified recombinant β2GPI (4).

FIG. 4. Amyloid-like cross-β structure in alkylated murine serum albumin and in heat-denatured ovalbumin, murine serum albumin, human glucagon and Etanercept.

A. Plg-activation assay with Pls activity read-out using chromogenic substrate S-2251. Activating properties of reduced and alkylated murine serum albumin (alkyl-MSA) and heat-denatured ovalbumin (dOVA) are compared with amyloid γ-globulins (positive control), buffer (negative control), and native albumin and ovalbumin (nMSA, nOVA). B. Thioflavin T fluorescence assay with native and denatured MSA and OVA. C. tPA activation assay for comparison of reduced and alkylated MSA and heat-denatured MSA. D. ThT fluorescence assay with reduced/alkylated MSA and heat-denatured MSA. E. tPA activation assay with concentration series of heat/acid denatured glucagon. F. ThT fluorescence assay with native and heat/acid denatured glucagon. G. Comparison of the tPA activating properties of heat-denatured Etanercept, native Etanercept and reduced/alkylated Etanercept. H. ThT fluorescence of native and heat-denatured Etanercept. I. TEM image of heat-denatured ovalbumin. The scale bar represents 200 nm. J. TEM image of heat/acid-denatured glucagon. The scale bar represents 1 μM. K. ThT fluorescence assay showing that filtration through a 0.2 μm filter of denatured OVA does not influence the fluorescence enhancing properties.

FIG. 5. Binding of tPA F-EGF, fibronectin F4,5 and BiP to misfolded proteins.

A. Binding of BiP, fibronectin F4-5 and tPA F-EGF to BSA-AGE, as observed by ELISA, detected using Ni-NTA-HRP. The finger domains show high affinity binding, whereas BiP shows low affinity for BSA-AGE in this set-up. B-C. Affinity of BiP for misfolded proteins tested in an ELISA (detection anti-FLAG-HRP). BiP has a high affinity AGEs (BSA-AGE (B.) and Hb-AGE (C.)), but not for their freshly dissolved controls. D.-H. Binding of fibronectin F4,5 (Fn F4,5) to several (mis)folded proteins as observed in an ELISA set-up (detection anti-FLAG HRP). Fn F4,5 binds to most misfolded proteins with higher (AGEs, D., E.) or lower (heat-denatured OVA (F.) or denatured γ-globulins (G.)) affinity, without recognising their native controls. H. Fn F4,5 recognises both native and alkyl-lysozyme with medium affinity. I. Binding of tPA-F EGF to heat-denatured and native OVA, as tested in an ELISA setup.

FIG. 6: Extraction with misfolded protein affinity matrices of BSA-AGE from solution. BSA-AGE at 0.5 μg/ml in PBS, 256-fold diluted serum in PBS and 512-fold diluted plasma in PBS, all in the presence of 0.1% Tween20 and 20 mM imidazole, was incubated with empty control Ni-Sepharose beads or indicated misfolded protein binding moieties tPA F-EGF, BiP, sRAGE and Fn F4,5, all bound to Ni-Sepharose. The content of BSA-AGE before and after the incubation was assessed by applying the solutions in a sandwich assay with anti-AGE antibody and anti-albumin antibody. Background signals when using PBS, serum or plasma without the BSA-AGE spike were subtracted from the depicted signals. A. Depletion of PBS from BSA-AGE. B. Depletion of diluted serum from BSA-AGE. C. Depletion of diluted plasma from BSA-AGE.

FIG. 7. Effect of depletion of a solution from misfolded protein on activation of platelets.

A. Representative standard curve of tPA F-biotin in a direct ELISA for detection of tPA F-biotin in solution. Shown is the tPA F-biotin supernatant before and after contacting to Streptavidin-Sepharose beads for coupling purposes. B. Contacting buffer or diluted plasma with a 1 μg/ml BSA-AGE spike with tPA F-biotin—Streptavidin-Sepharose results in depletion of the solutions from BSA-AGE, as determined in a sandwich ELISA using coated anti-AGE antibody and anti-albumin detecting antibody. C. Platelet aggregation is induced by 512-fold diluted plasma with 250 μg/ml BSA-AGE spike. After contacting the diluted plasma with BSA-AGE with tPA F-biotin—Streptavidin-Sepharose, platelet aggregating properties is strongly reduced.

FIG. 8. Binding of recombinant human extracellular cluster IV fragment of low density lipoprotein receptor related protein to misfolded amyloid-like glycated protein.

A. LRP cluster IV binds specifically and in a dose-dependent manner to immobilized amyloid-like misfolded glycated albumin and glycated haemoglobin. B-E. ELISA showing the influence of tPA and K2P tPA (B.), ThT (C.), Congo red (D.) anf ThS (E.) on binding of LRP cl-IV to immobilized amyloid-like misfolded BSA-AGE.

FIG. 9. Misfolded amyloid-like β2-glycoprotein I elicits a humoral auto-immune response in mice.

A. Generation of plasmin from tPA/plasminogen is accelerated when β2gpi is exposed to cardiolipin (CL-β2gpi), which results in amyloid-like properties in β2gpi. B. Alkylation of cysteine residues in β2gpi induces amyloid-like protein conformation, as shown by enhanced Thioflavin T fluorescence. C. Alkylation of β2gpi results in amyloid-like properties when the ability to activate tPA/plasminogen is considered. In the assay 100 μg/ml alkyl-β2gpi is compared with 100 μg/ml native β2gpi and 100 μg/ml amyloid-like misfolded γ-globulins (positive assay control). Negative control was H₂O.

FIG. 10. Amyloid-like properties of protein therapeutics increase during storage within expiry limits, under conditions as defined by manufacturer information.

Biopharmaceutical preparations were tested (at 25 μg/ml protein) twice over several months for their capacity to enhance ThT and Congo red fluorescence. Samples were measured in triplicate at each time point.

FIG. 11. Various biopharmaceuticals adopt amyloid-like properties after exposure to conditions of stress.

Etanercept, Glucagon, Abciximab and Infliximab were exposed to denaturing conditions (see materials & methods) and subsequently analyzed for the presence of amyloid-like properties, using ThT-fluorescence (A.) and tPA activation assay (B.; expressed as percentage of standardized positive control). N=native, D=denatured.

FIG. 12. Interaction of ellagic acid with misfolded proteins.

A. Ellagic acid was co-incubated with 0.5 nM tPA and binding to immobilized Hb-AGE was assessed in an ELISA. [Hb-AGE] is 1.25 μg/nl. [ThT] is 1 mM positive control for stimulated tPA binding). [Congo red] is 0.5 mM (positive control for inhibited tPA binding). B. Binding of tPA to misfolded ovalbumin, after pre-incubation of immobilized misfolded ovalbumin with ellagic acid at 500 μg/ml. C. Binding of tPA to immobilized Hb-AGE, that was first overlayed with concentration series of ellagic acid. D. Binding of tPA to immobilized Aβ, that was first overlayed with concentration series of ellagic acid. E. Binding of tPA to immobilized misfolded ovalbumin, that was first overlayed with concentration series of ellagic acid.

FIG. 13. Amyloid-ike conformations are detected on activated blood platelets and contribute to platelet aggregation.

A-B. Analysis of amyloid formation during adhesion of platelets in whole blood to collagen (A) or von Willebrand factor (B) under flow for 5 minutes. Samples were stained with the amyloid specific dye Congo red. Images are at 100× magnification. Platelets show the characteristic spreading on collagen or vWf. C-D. FACS analysis of platelets stimulated with (D) or without (C) thrombin (1 min, 37° C.) in the presence of EDTA. The fluorescent amyloid dye Thioflavin T was used to detect amyloid on the surface of platelets. E. Washed platelets were exposed to thrombin activating peptide (TRAP) in the presence or absence of ThT (200 μM), Congo Red (200 μM) or tPA (1 μM) where indicated. Platelet aggregation was assessed by light scattering. F. Activation of platelets in the presence of TRAP, indo and AR, with or without tPA. Indo: indomethacin (aspirin-like), AR-C6993MX (clopidogrel-like). TPA further decreases the level of TRAP-induced platelet activation, that is suppressed by indo and AR. G. Thioflavin T fluorescence measurement at t=0 and t=2h15′ after incubation of platelets in HEPES-Tyrode or with ionophore A23187. H. Congo red fluorescence measurement at t=0 and t=2h15′ after incubation of platelets in HEPES-Tyrode or with ionophore A23187. I. tPA/plasminogen activation assay with 50.000 platelets/μl of control platelets and platelets exposed to ioniphore A23187.

### REFERENCE LIST

1. Cleland, J. L., Powell, M. F. & Shire, S. J. The development of stable protein formulations: a close look at protein aggregation, deamidation, and oxidation. Crit Rev. Ther. Drug Carrier Syst. 10, 307-377 (1993).
2. Wang, W. Protein aggregation and its inhibition in biopharmaceutics. Int. J. Pharm. 289, 1-30 (2005).
3. Krishnamurthy, R. & Manning, M. C. The stability factor: importance in formulation development. Curr. Pharm. Biotechnol. 3, 361-371 (2002).
4. Hermeling, S., Crommelin, D. J., Schellekens, H. & Jiskoot, W. Structure-immunogenicity relationships of therapeutic proteins. Pharm. Res. 21, 897-903 (2004).
5. Bouma, B. et al. Glycation induces formation of amyloid cross-beta structure in albumin. J. Biol. Chem. 278, 41810-41819 (2003).
6. Nilsson, M. R. Techniques to study amyloid fibril formation in vitro. Methods 34, 151-160 (2004).
7. Bucciantini, M. et al. Inherent toxicity of aggregates implies a common mechanism for protein misfolding diseases. Nature 416, 507-511 (2002).
8. Cribbs, D. H., Azizeh, B. Y., Cotman, C. W. & LaFerla, F. M. Fibril formation and neurotoxicity by a herpes simplex virus glycoprotein B fragment with homology to the Alzheimer's A beta peptide. Biochemistry 39, 5988-5994 (2000).
9. Kayed, R. et al. Common structure of soluble amyloid oligomers implies common mechanism of pathogenesis. Science 300, 486-489 (2003).
10. Kranenburg, O. et al. Recombinant endostatin forms amyloid fibrils that bind and are cytotoxic to murine neuroblastoma cells in vitro. FEBS Lett. 539, 149-155 (2003).
11. Tucker, H. M., Kihiko-Ehmann, M., Wright, S., Rydel, R. E. & Estus, S. Tissue plasminogen activator requires plasminogen to modulate amyloid-beta neurotoxicity and deposition. J. Neurochem. 75, 2172-2177 (2000).
12. Klein, W. L., Irafft, G. A. & Finch, C. E. Targeting small Abeta oligomers:

the solution to an Alzheimer's disease conundrum? Trends Neurosci. 24, 219-224 (2001).
13. Buxbaum, J. N. Diseases of protein conformation: what do in vitro experiments tell us about in vivo diseases? Trends Biochem. Sci. 28, 585-592 (2003).
14. Reixach, N., Deechongkit, S., Jiang, X., Kelly, J. W. & Buxbaum, J. N. Tissue damage in the amyloidoses: Transthyretin monomers and nonnative oligomers are the major cytotoxic species in tissue culture. Proc. Natl. Acad. Sci. U.S.A. 101, 2817-2822 (2004).
15. O'Nuallain, B. & Wetzel, R. Conformational Abs recognizing a generic amyloid fibril epitope. Proc. Natl. Acad. Sci. U.S.A. 99, 1485-1490 (2002).
16. Kranenburg, O. et al. Tissue-type plasminogen activator is a multiligand cross-beta structure receptor. Curr. Biol. 12, 1833-1839 (2002).
17. Schielen, J. G., Adams, H. P., Voskuilen, M., Tesser, G. J. & Nieuwenhuizen, W. Structural requirements of position A alpha-157 in fibrinogen for the fibrin-induced rate enhancement of the activation of plasminogen by tissue-type plasminogen activator. Biochem. J. 276, 655-659 (1991).
18. Thomassen, Y. E., Meijer, W. J., Sierkstra, L. & Verrips, C. T. Large-scale production of VHH antibody fragments by Saccharomyces cerevisiae. Enzyme and Microbial. Techology 30, 273-278 (2002).
19. Hackeng, T. M., Griffin, J. H. & Dawson, P. E. Protein synthesis by native chemical ligation: expanded scope by using straightforward methodology. Proc. Natl. Acad. Sci. U.S.A. 96, 10068-10073 (1999).
20. de Laat, B., Derksen, R. H., Urbanus, R. T. & de Groot, P. G. IgG antibodies that recognize epitope Gly40-Arg43 in domain I of {beta}2-glycoprotein I cause LAC and their presence correlates strongly with thrombosis. Blood., (2004).
21. Horbach, D. A., van Oort, E., Donders, R. C., Derksen, R. H. & de Groot, P. G. Lupus anticoagulant is the strongest risk factor for both venous and arterial thrombosis in patients with systemic lupus erythematosus. Comparison between different assays for the detection of antiphospholipid antibodies. Thromb. Haemost. 76, 916-924 (1996).
22. Horbach, D. A., van Oort, E., Tempelman, M. J., Derksen, R. H. & de Groot, P. G. The prevalence of a non-phospholipid-binding form of beta2-glycoprotein I in human plasma—consequences for the development of anti-beta2-glycoprotein I antibodies. Thromb. Haemost. 80, 791-797 (1998).
23. Onoue, S. et al. Mishandling of the therapeutic peptide glucagon generates cytotoxic amyloidogenic fibrils. Pharm. Res. 21, 1274-1283 (2004).
24. Bouma, B. et al. Adhesion mechanism of human beta(2)-glycoprotein I to phospholipids based on its crystal structure. EMBO J. 18, 5166-5174 (1999).
25. Subang R, Levine J S, Janoff A S et al. Phospholipid-bound beta 2-glycoprotein I induces the production of anti-phospholipid antibodies. J Autoimmun. 2000;15:21-32.

1. A method for detecting a protein and/or peptide comprising a cross-β structure in an aqueous solution comprising a protein, said method comprising:
a. contacting said aqueous solution with at least one cross-β structure-binding compound resulting in a bound protein and/or peptide comprising a cross-β structure, and
b. detecting whether bound proteins and/or peptides comprising a cross-β structure are present in said aqueous solution.

2. The method according to claim 1, wherein said cross-β structture-binding compound is a compound selected from the group of compounds identified in table 1, table 2, and table 3.

3. The method according to claim 1, wherein said aqueous solution further comprises a detergent, a food, a food supplement, a cell culture medium, a commercially available protein, protein/peptide solutions used for research purposes, blood, blood products, a cosmetic product, and/or a cell.

4. A method for controling a manufacturing process, and/or storage process of an aqueous solution comprising a protein, said method comprising:
a. contacting said aqueous solution with at least one cross-β structure-biniding compound resulting in a bound protein or peptide comprising a cross-β structure. and
b. detecting whether bound proteins and/or peprides comprising a cross-β structure are present in said aqueous solution at various stages of said manufacturing and/or storage process.

5. A method for removing a protein and/or peptide comprising a cross-β structure from an aqueous solution comprising a protein, said method comprising:
a. contacting said aqueous solution with at least one cross-β structure-binding compound resulting in a hound protein and/or peptide comprising a cross-β structure,
b. allowing binding of said protein and/or peptide comprising a cross-β structure to said cross-β structure-binding compound, and
c. separating said bound protein and/or peptide comprising a cross-β structure from said aqueous solution comprising a protein.

6. The method according to claim 5, wherein said cross-β structure-binding compound is a compound selected from the group of compounds identified in table 1, table 2, and table 3.

7. The method according to claim 5, wherein said cross-β binding compound is bound to a second compound.

8. The method according to claim 7, wherein said second compound is bound to a solid phase.

9. A method for decreasing and/or preventing undesired side effects of an aqueous solution comprising a protein and/or increasing the specific activity per gram protein of the aqueous solution, said method comprisin-g
detecting and removing any unfolded protein or peptide and/or aggregated protein or peptide and/or multimerized protein or peptide comprising a cross-β structure from said aqueous solution with the method according to claim 1.

10. An aqueous solution comprising a protein, obtainable by the method according to claim 1.

11. A kit for carrying out a method for binding a peptide having a cross-β structure that may be found in an aqueous solution, the kit comprising;
means for contacting the aqueous solution with a cross-β structure-binding compound, and
a cross-β structure-binding compound.

12. A method for detecting a cell comprising a protein and/or peptide with cross-β structure on its surface in a collection of cells, said method comprising.
contacting said cell with a cross-β structure-binding molecule, and
measuring binding of said cross-β structure-bindina molecule to said cell.

13. A method for removing a cell comprising a protein and/or peptide with cross-β structure on its surface from a collection of cells. said method comprisinr
contacting said cell with a cross-β structure-binding molecule, and
binding said cross-β strcture-binding molecule to a solid surface.

14. The method according to claim 1, wherein said cross-β structure binding compound comprises ellagic acid.

15. The method according to claim 2, wherein said aqueous solution further comprises a detergent, a food, a food supplement, a cell culture medium, a commercially available protein, protein/peptide solutions used for research purposes, blood, blood products, a cosmetic product, and/or a cell.

16. The method according to claim 6. wherein the cross-β binding compound is bound to a second compound.

17. The method according to claim 16, wherein the second compound is bound to a solid phase.

18. The method according to claim 4, wherein said cross-β structure binding compound comprises ellagic acid.

19. The method according to claim 5, wherein said cross-β stricture binding compound comprises ellagic acid.

20. The method according to claim 12, wherein said cross-β structure binding compound comprises ellagic acid.
